(19) United States
(12) Patent Application Publication
(10) Pub. No.: US 2018/0140610 A1
(43) Pub. Date: May 24, 2018

(54) OPIPRAMOL PATCH

(71) Applicant: NeuroDerm, Ltd., Rehovot (IL)

(72) Inventors: Oron YACOBY-ZEEVI, Moshav Bitsaron (IL); Mara NEMAS, Gedera (IL)

(73) Assignee: NeuroDerm, Ltd., Rehovot (IL)

(21) Appl. No.: 15/511,428

(22) PCT Filed: Sep. 18, 2015
(86) PCT No.: PCT/IB2015/002139

§ 371 Date: Feb. 2, 2018

Related U.S. Application Data
(60) Provisional application No. 62/052,183, filed on Sep. 18, 2014.

(51) Int. Cl. A61K 31/55; A61K 9/70; A61K 47/12; A61K 47/10; A61K 47/22; A61K 47/14; A61K 47/26; A61M 35/00

(52) U.S. Cl. CPC A61K 31/55; A61K 9/7061; A61K 9/7053; A61K 47/12; A61M 35/00; A61K 47/22; A61K 47/14; A61K 47/26; A61K 47/10

(57) ABSTRACT

Disclosed herein are compositions that are useful in effecting the transdermal delivery of active agents such as opipramol. More particularly, the disclosed transdermal compositions include an active agent; one or more plasticizers; one or more penetration enhancers; a pressure-sensitive adhesive; and may include one or more hydrophilic polymers.

### CROSS REFERENCE TO RELATED APPLICATIONS

This application claims the benefit of and priority to U.S. Provisional Patent Application Ser. No. 62/052,183, filed Sep. 18, 2014, the contents of which are incorporated by reference herein.

### BACKGROUND

Active agents (for example, therapeutic agents such as drugs or immunologically active agents such as vaccines) are conventionally administered either orally or by injection. However, many active agents are completely ineffective or have radically reduced efficacy when orally administered since they either are not absorbed or are adversely affected before entering the bloodstream and thus do not possess the desired activity. On the other hand, the direct injection of active agents intravenously or subcutaneously, while assuring no modification of the agents during administration, can be invasive, painful, and often results in poor patient compliance.

Transdermal delivery of active agents, however, may result in systemic circulation of the active agent and can provide an alternative mode of administration. For example, transdermal delivery can potentially provide better drug bioavailability than oral administration, in part because such delivery bypasses not only the initial metabolism of the drug by the liver and the intestines but also the unpredictable absorption of the drug from the gastrointestinal tract. Transdermal delivery may result in more stable blood serum level of the drug (e.g., leading to a prolonged pharmacological effect that is similar to intravenous infusion), and can allow for easily adjustable dosing rate. For example, transdermal patches can be easily removed which results in rapid cessation of dosing and elimination of the drug from circulation. Finally, transdermal delivery typically results in greater patient compliance because it is non-invasive and can be easily administered.

The skin serves as a barrier to the penetration of many foreign substances. The feasibility of using transdermal delivery of active agents as a route of administration requires that a therapeutic rate of drug delivery through the skin be achieved.

However, it is often difficult to find compositions that include active agents and also are effective in penetrating the skin. Consequently, there remains a need for transdermal formulations that could deliver, at controlled rates, an active agent or a mixture thereof.

### SUMMARY

Provided herein are pharmaceutically acceptable transdermal compositions for the controlled administration of an active agent. Contemplated herein are transdermal drug compositions for the transdermal delivery of an active agent such as opipramol to a patient, such drug compositions comprising:
a. a plasticizer,
b. a penetration enhancer,
c. a pressure-sensitive adhesive (PSA), and
d. opipramol or a pharmaceutically acceptable salt thereof,
   wherein said drug composition can form an adhesive layer. The compositions may optionally include a hydrophilic polymer.

In one aspect, the present disclosure relates to a transdermal delivery device for the transdermal delivery of opipramol comprising:
a. an inert layer detachable when used;
b. at least one adhesive layer comprising a transdermal drug composition described herein, wherein the adhesive layer is directly affixed to a surface of the inert layer; and
c. a backing layer, coated over the adhesive layer.

In another aspect, the present disclosure relates to method of treating a patient having a disorder selected from the group consisting of central nervous system (CNS) disorders, peripheral nervous system disorders, factitious disorders, somatoform disorders, inflammatory disorders, and pain-related disorders, said method comprising the steps of:
a. providing a transdermal delivery device described herein;
b. placing an adhesive layer of the device against the skin of the patient, thereby providing an amount of opipramol effective to treat the disorder.

In another aspect, the present disclosure relates to a method of preventing, treating, or suppressing tobacco or nicotine dependence or usage in a patient, said method comprising the steps of:
a. providing a transdermal delivery device described herein;
b. placing the adhesive layer of the device against the skin of the patient, thereby providing an amount of opipramol effective to prevent, treat, or suppress the tobacco or nicotine dependence or usage.

### BRIEF DESCRIPTION OF THE DRAWINGS

FIG. 1 depicts the influence of different opipramol concentrations on the transdermal delivery of opipramol through pig skin ex vivo.

FIG. 2 depicts the influence of different opipramol concentrations on the transdermal delivery of opipramol through pig skin ex vivo.

FIG. 3 depicts a graph showing the effect of oleic acid in comparison to azelaic acid with and without Eudragit® on the transdermal delivery of opipramol through pig skin ex vivo through patches comprising 0.2% Tween® 80.

FIG. 4 depicts a graph showing the effect of oleic acid in comparison to azelaic acid with and without Eudragit® on the transdermal delivery of opipramol through pig skin ex vivo through patches comprising 5% Tween® 80.

FIG. 5 depicts a graph showing the effect of oleic acid in comparison to oleyl alcohol on the transdermal delivery of opipramol through pig skin ex vivo through patches comprising 5% oleic acid and different concentrations of Eudragit®.

FIG. 6 depicts the effect of Transcutol® on the transdermal delivery of opipramol through pig skin ex vivo through patches with and without Eudragit®.

FIG. 7 depicts the effect of Eudragit® compounds on the transdermal delivery of opipramol through pig skin ex vivo through patches comprising 1% Tween® 80.

FIG. 8 depicts the effect of Eudragit® compounds on the transdermal delivery of opipramol through pig skin ex vivo through patches comprising 0.2% Tween® 80.

FIG. 9 depicts the effect of Azone® on the transdermal delivery of opipramol through pig skin ex vivo.

FIG. 10 depicts the effect of Azone® on the transdermal delivery of opipramol through pig skin ex vivo through patches comprising varying concentrations of Tween® 80.

FIG. 11 depicts the effect of IPM on the transdermal delivery of opipramol through pig skin ex vivo through patches with or without Azone®.

FIG. 12 depicts the effect of Plasdone™ S-630 on the transdermal delivery of opipramol through pig skin ex vivo through patches comprising varying concentrations of Tween® 80 and Azone®.

FIG. 13 depicts the effect of N-methylpyrrolidone (NMP) on the transdermal delivery of opipramol through pig skin ex vivo.

FIG. 14 depicts a graph of the concentration of opipramol in pig plasma.

FIG. 15 depicts the plasma levels of opipramol in small (FIG. 15A) vs. large pigs (FIG. 15B).

FIGS. 16 to 22 depict the delivery rates of opipramol by patches containing differing types of layers.

Unless indicated otherwise, all amounts indicated in the above Figures are weight percent.

### DETAILED DESCRIPTION

### Definitions

For convenience, certain terms used in the specification, examples, and appended claims are collected in this section.

The term “continuously” or “continuous delivery” as used herein refers to a drug delivered substantially slowly and substantially uninterrupted for e.g. 2, 3, 8, 12, or more hours or even 1, 2, 3, 5, 7, or 10 or more days. In some embodiments, the term continuously refers to delivery of a drug or agent that is substantially longer as compared to bolus single or multiple doses. For this purpose, the transdermal patches according to the present disclosure are suitable.

The terms “cognitive disorder” or “cognitive dysfunction” refer to mental conditions that cause patients to have difficulty in thinking with symptoms generally marked by impaired attention, perception, reasoning, memory and judgment. One type of cognitive disorder is dementia, which is characterized by gradual impairment of multiple cognitive abilities including memory, language and judgment. Impairment of cognitive abilities can be caused by, or associated with, neurodegenerative or neurological diseases, disorders or conditions such as Alzheimer's disease, Parkinson's disease, Huntington's disease, Lewy body disease, Pick's disease, Jakob-Creutzfeld disease, multiple sclerosis, anxiety, depression, schizophrenia, limbic encephalitis, normal pressure hydrocephalus, age-related memory impairment; brain damage caused by stroke, brain injuries and vascular dementia; post-trauma injury; infectious diseases such as neurosyphilis, acquired immune deficiency syndrome (AIDS), fungal infections, tuberculosis; drug intoxication such as alcohol, nicotine, cannabis, and cocaine addiction or heavy metal exposure. Attention deficit disorder (ADD) and attention deficit hyperactivity disorder (ADHD) are types of cognitive dysfunction found both in children and adults.

The term “treating” is used herein to denote treating the disease, disorder or condition, or ameliorating, alleviating, reducing, or suppressing symptoms of the disease, or slowing or stopping the progress of the disease. Thus, in some embodiments, administration of the composition or combination of the present disclosure may ameliorate, alleviate or reduce the cognitive disorder symptoms in dementia associated with the diseases, disorders and conditions as mentioned above.

The term “therapeutically effective amount” refers to the amount of an active ingredient, or combination of active ingredients, that will elicit the biological or medical response that is being sought by the researcher, veterinarian, medical doctor or other clinician. Alternatively, a therapeutically effective amount of an active ingredient is the quantity of the compound required to achieve a desired therapeutic and/or prophylactic effect, such as the amount of the active ingredient that results in the prevention of or a decrease in the symptoms associated with the condition (for example, to meet an end-point).

The terms “pharmaceutically acceptable” or “pharmacologically acceptable” refer to molecular entities and compositions that do not produce an adverse, allergic or other untoward reaction when administered to an animal, or to a human, as appropriate. The term “pharmaceutically acceptable carrier” includes any and all solvents, dispersion media, coatings, antibacterial and antifungal agents, isotonic and absorption delaying agents and the like. The use of such media and agents with pharmaceutical active agents is well known in the art. In some embodiments, supplementary active ingredients can also be incorporated into the compositions.

The terms “carrier” or “vehicle” as used herein refer to carrier materials suitable for transdermal drug administration. Contemplated carriers and/or vehicles include any such materials known in the art, which are substantially nontoxic and/or do not interact with other components of a pharmaceutical formulation or drug delivery system in a deleterious manner. Examples of specific suitable carriers and vehicles for use herein include water, propylene glycol, mineral oil, silicone, inorganic gels, aqueous emulsions, liquid sugars, waxes, petroleum jelly, and/or other oils and polymeric materials.

The term “transdermal” refers generally to passage of an agent across the skin layers. For example, the term “transdermal” may refer to delivery of an agent (e.g., a vaccine or a drug) through the skin to the local tissue or systemic circulatory system without substantial cutting or penetration of the skin, such as cutting with a surgical knife or piercing the skin with a hypodermic needle. The term “transdermal delivery” refers to drug delivery across the skin, usually accomplished without breaking the skin. Transdermal delivery includes delivery via passive diffusion.

The terms “penetration enhancement” or “permeation enhancement” as used herein refer to an increase in the permeability of skin to a pharmacologically active agent, i.e., so as to increase the rate at which the active agent permeates through the skin and enters the bloodstream. The enhanced permeation effected through the use of skin permeation enhancers, for example, through the use of a composition disclosed herein, can be observed by e.g., measuring the rate of diffusion of drug ex vivo, i.e., through animal or human skin using a diffusion cell apparatus, or in vivo, as described in the examples herein.

The terms, “individual,” “patient,” or “subject” are used interchangeably herein and include any mammal, including animals, for example, primates, for example, humans, and other animals, for example, dogs, cats, swine, cattle, sheep, rodents, and horses. The compositions disclosed herein can be administered to a mammal, such as a human, but can also be other mammals, for example, an animal in need of veterinary treatment, for example, domestic animals (for example, dogs, cats, and the like), zoo and wild animals, farm animals (for example, cows, sheep, pigs, horses, and the like) and laboratory animals (for example, rats, mice, guinea pigs, and the like). The subject may be in need of treatment by delivery of a therapeutic agent, for example, transcutaneous delivery of a vaccine or transdermal delivery of a drug.

As used herein, the term “organic acid” refers to carbon-containing acidic compounds (e.g. carbon-containing phosphoric acids or sulfonic acids and carboxylic acids) suitable for use in transdermal compositions. Such organic acids include, without limitation, ascorbic acid, tartaric acid, malic acid, succinic acid, fumaric acid, citric acid, lactic acid, salicylic acid and salicylates (including their methyl, ethyl, and propyl glycol derivatives), among others.

The compositions of the present disclosure may optionally include one or more polyols. As used herein, the term “polyol” refers to saturated or unsaturated, branched or unbranched C₃-C₆₀, C₃-C₃₀, or C₃-C₂₀ carbon chains functionalized with at least two hydroxyl (—OH) groups and includes without limitation propylene glycol, butylene glycol, polyethylene glycol, ethylene glycol, diethylene glycol, triethylene glycol, dipropylene glycol, ethoxydiglycol, pentylene glycol, glycerol, propanediol, butanediol, pentanediol, sorbitol, sucrose, mannitol, trehalose, hexanetriol, and glycerin, among others.

The compositions of the disclosure can also include natural fats and oils. As used herein, the term “natural fat or oil” or “edible fat or oil” is intended to include fats, oils, essential oils, essential fatty acids, non-essential fatty acids, phospholipids, and combinations thereof. These natural fats and oils can provide a source of essential and non-essential fatty acids to those found in the skin's natural barrier. Suitable natural fats or oils can include without limitation citrus oil, olive oil, avocado oil, apricot oil, babassu oil, borage oil, camellia oil, canola oil, castor oil, coconut oil, corn oil, cottonseed oil, emu oil, evening primrose oil, hydrogenated cottonseed oil, hydrogenated palm kernel oil, maleated soybean oil, meadowfoam oil, palm kernel oil, peanut oil, rapeseed oil, grapeseed oil, safflower oil, sphingolipids, seed almond oil, tall oil, lauric acid, palmitic acid, stearic acid, linoleic acid, stearyl alcohol, lauryl alcohol, myristyl alcohol, behenyl alcohol, rose hip oil, calendula oil, chamomile oil, eucalyptus oil, juniper oil, sandlewood oil, tea tree oil, sunflower oil, soybean oil, and combinations thereof.

The compositions of the present disclosure may optionally include one or more amides, e.g. cyclic amides or N,N-dialkylamides. Examples of suitable amides include urea, N,N-dialkylamides such as N,N-dimethylacetamide, N,N-diethyltoluamide, N,N-dimethylformamide (DMF), N,N-dimethyloctamide, and N,N-dimethyldecamide, biodegradable cyclic ureas (e.g., 1-alkyl-4-imidazoline-2-one), pyrrolidone derivatives, biodegradable pyrrolidone derivatives (e.g., fatty acid esters of N-(2-hydroxyethyl)-2-pyrrolidone), hexamethylenelauramide and its derivatives, diethanolamine, and triethanolamine. Examples of cyclic amides include pyrrolidone derivatives such as 1-methyl-2-pyrrolidone, 2-pyrrolidone, 1-lauryl-2-pyrrolidone, 1-methyl-4-carboxy-2-pyrrolidone, 1-hexyl-4-carboxy-2-pyrrolidone, 1-lauryl-4-carboxy-2-pyrrolidone, 1-methyl-4-methoxycarbonyl-2-pyrrolidone, 1-hexyl-4-methoxycarbonyl-2-pyrrolidone, 1-lauryl-4-methoxycarbonyl-2-pyrrolidone, N-cyclohexylpyrrolidone, N-dimethylaminopropylpyrrolidone, N-cocoalkypyrrolidone, N-tallowalkylpyrrolidone, and N-methylpyrrolidone (NMP). Further examples of cyclic amides include 1-dodecylazacycloheptane-2-one (i.e., Azone®), 1-geranylazacycloheptan-2-one, 1-farnesylazacycloheptan-2-one, 1-geranylgeranylazacycloheptan-2-one, 1-(3,7-dimethyloctyl)azacycloheptan-2-one, 1-(3,7,11-trimethyldodecyl)azacyclohaptane-2-one, 1-geranylazacyclohexane-2-one, 1-geranylazacyclopentan-2,5-dione and 1-farnesylazacyclopentan-2-one.

Compositions of the present disclosure may optionally include one or more fatty alcohols. As used herein, the term “fatty alcohol” may refer to saturated or unsaturated, branched or unbranched C₄-C₆₀, C₇-C₃₀, or C₈-C₂₀ carbon chains functionalized with an alcohol (—OH). Fatty alcohols contemplated for use in disclosed compositions, include, but are not limited to, 1-octanol, 2-octanol, 3-octanol, 4-octanol, hexanol, heptanol, nonanol, decanol (capric alcohol), undecanol, dodecanol (lauryl alcohol), 2-ethyl hexanol, pelargonic alcohol, myristyl alcohol, cetyl alcohol, palmitoleyl alcohol, octadeconal (stearyl alcohol), isostearyl alcohol, isolauryl alcohol, isomyristyl alcohol, isopalmityl alcohol, isostearyl alcohol, elaidyl alcohol, oleyl alcohol, linoleyl alcohol, elaidolinoleyl alcohol, linoleynyl alcohol, elaidolinolenyl alcohol, ricinoleyl alcohol, arachidyl alcohol, behenyl alcohol, erucyl alcohol, lignoceryl alcohol, ceryl alcohol, montanyl alcohol, myricyl alcohol, geddyl alcohol, cetearyl alcohol, and mixtures thereof.

Compositions of the present disclosure may optionally include one or more fatty acid esters. As used herein, the term “fatty acid ester” may refer to saturated or unsaturated, branched or unbranched C₄-C₆₀, C₇-C₃₀, or C₈-C₂₀ carbon chains functionalized with an ester moiety. Contemplated fatty acid esters include, but are not limited to, lauroglycol, methyl laurate, ethyl oleate, propylene glycol monolaurate, propylene glycerol dilaurate, glycerol monolaurate, glycerol monooleate, sorbitan monooleate, isopropyl palmitate, methyl propionate, monoglycerides, sorbitan monolaurate, isopropyl n-decanoate, and oetyldodecyl myristate, and mixtures thereof.

Compositions of the present disclosure may optionally include one or more fatty acids. As used herein, the term “fatty acid” may refer to saturated or unsaturated, branched or unbranched C₄-C₆₀, C₇-C₃₀, or C₈-C₂₀ carbon chains functionalized with a carboxylic acid. Contemplated fatty acids include, but are not limited to, oleic acid, alkanoic acids, capric acid, hexanoic acid, lactic acid, lauric acid, linoleic acid and mixtures thereof.

Contemplated transdermal compositions may include a terpene, which as used herein refers to a nonaromatic compound found in essential oils, which may be extracted from flowers, fruits, and other natural products. Exemplary terpenes include, but are not limited to, d-limonene, dipentene (d/l-limonene), α-pinene, terpinene, β-mircene, p-cimene, α-pinene, α-phellandrene, citronellolio, geraniale (citrale), nerol, beta-carotene, menthol, geraniol, farnesol, phytol, their homologs, derivatives, enantiomers, isomers including constitutional isomers, stereoisomerisms, regioisomers, and geometric isomers, and any combinations thereof. Suitable terpenes include alcohols (e.g. α-terpineol, terpinen-4-ol, carvol, etc.), ketones (e.g., carvone, pulegone, piperitone, menthone, etc.), oxides (e.g., cyclohexene oxide, limonene oxide, α-pinene oxide, cyclopentene oxide, 1,8-cineole, etc.), and oils (e.g., ylang ylang, anise, chenopodium, eucalyptus, peppermint, etc.).

### Compositions

The present disclosure relates, in part, to transdermal drug compositions for the transdermal delivery of an active agent to a patient. Disclosed herein are transdermal compositions that may be part of, for example, a transdermal patch, ointment, cream, gel, lotion, spray, or other transdermal solution or suspension. For example, for transdermal delivery, a transdermal patch that includes a disclosed composition is contemplated, and may include a single layer adhesive patch, a multi-layer adhesive patch, a reservoir patch, a matrix patch, a microneedle patch, or an iontophoretic patch, which typically requires applying a direct current. In some embodiments, contemplated transdermal patches may be adapted for continuous release.

Contemplated transdermal drug delivery systems can, in some embodiments, rely on passive, chemical diffusion as opposed to physical, electrical, or mechanical based approaches. For example, passive transdermal systems may have a drug reservoir containing a high concentration of drug adapted to contact the skin where the drug diffuses through the skin and into the body tissues or bloodstream of a patient.

Thus, in one aspect, the present disclosure provides a transdermal drug composition for the transdermal delivery of opipramol to a patient, the drug composition comprising:
a. a plasticizer,
b. a penetration enhancer,
c. a pressure-sensitive adhesive (PSA), and
d. opipramol or a pharmaceutically acceptable salt thereof,
   wherein said drug composition can form an adhesive layer.

In certain embodiments, the transdermal drug composition further comprises a hydrophilic polymer (e.g., a polymethacrylate polymer, a polyvinylpyrrolidone polymer, or a combination thereof).

In certain embodiments:
a. the polymethacrylate polymer is selected from the group consisting of poly(methacrylic acid-co-methyl methacrylate) 1:1 (Eudragit® L100), poly(ethyl acrylate-co-methyl methacrylate-co-trimethylammonioethyl methacrylate chloride) 1:2:0.2 (Eudragit® RL100), poly(methacrylic acid-co-methyl methacrylate) 1:2 (Eudragit® S100), poly(butyl methacrylate-co-(2-dimethylaminoethyl) methacrylate-co-methyl methacrylate) 1:2:1 (Eudragit® E100), poly(methacylic acid-co-ethyl acrylate) 1:1 (Eudragit® L100-55), poly(butyl methacrylate-co-(2-dimethylaminoethyl) methacrylate-co-methyl methacrylate) 1:2:1 (Eudragit® E PO), poly(ethyl acrylate-co-methyl methacrylate-co-trimethylammonioethyl methacrylate chloride) 1:2:0.2 (Eudragit® RL PO), and poly(ethyl acrylate-co-methyl methacrylate-co-trimethylammonioethyl methacrylate chloride) 1:2:0.1 (Eudragit® RS PO); and
b. the polyvinylpyrrolidone polymer is selected from the group consisting of a 60:40 linear random copolymer of vinyl pyrrolidone and vinyl acetate (Plasdone™ S-630) and polyvinylpyrrolidone of average molecular weight from about 4,000 to about 58,000.

In certain embodiments, the plasticizer is selected from the group consisting of a fatty alcohol, a citric acid alkyl ester, a glycerol ester, phthalic acid alkyl ester, a sebacic acid alkyl ester, a sucrose ester, a sorbitan ester, an acetylated monoglyceride, a polyol, a fatty acid of 4-15 carbons, a fatty acid ester, a poloxamer, a mono- or di-glyceride of edible fats or oils, a glyceride, a polyethylene glycol (PEG), a sorbitan ester, a polysorbate, a disaccharide, and 2-(2-ethoxyethoxy)ethanol (Transcutol®), or a combination thereof. In particular embodiments, the plasticizer is Transcutol®.

In certain embodiments, the fatty alcohol is oleyl alcohol; and/or the polyol is selected from the group consisting of glycol, glycerol, propylene glycol, sorbitol, and mannitol; and/or the glycerol ester is glyceryl triacetate; the PEG has a molecular weight from about 200 to about 20,000; and/or the phthalic acid alkyl ester is diethyl phthalate or dibutyl phthalate; and/or the disaccharide is sucrose; and/or the fatty acid is lauric acid; and/or the sebacic acid alkyl ester is dibutyl sebacate; and/or the polysorbate is selected from the group consisting of polysorbate 20 (Tween® 20), polysorbate 40 (Tween® 40), polysorbate 60 (Tween® 60), and polysorbate 80 (Tween® 80); and/or or the sorbitan ester is selected from the group consisting of sorbitan laurate (Span® 20), sorbitan monopalmitate (Span® 40), sorbitan stearate (Span® 60), sorbitan tristearate (Span® 65), sorbitan monooleate (Span® 80), and sorbitan trioleate (Span® 85). In particular embodiments, the sorbitan ester is polysorbate 80.

In certain embodiments, said penetration enhancer is selected from the group consisting of a C₁-C₁₂ alcohol or ester, a C₂-C₃₀ diol, a C₃-C₃₀ polyol, a fatty alcohol, a fatty acid, a fatty acid ester, a polyoxyethylene fatty acid ester, a cyclic or N,N-dimethyl amide, a sorbitan monoester, a polyethylene glycol ether, a biodegradable cyclic urea, a polysaccharide, a terpene or essential oil, a surfactant, a sulfoxide, and a fatty acid or polyoxyethylene triglyceride, or a combination thereof.

In certain embodiments, the C₁-C₁₂ alcohol or ester is selected from the group consisting of ethanol, propanol, butanol, 2-butanol, pentanol, 2-pentanol, hexanol, heptanol, octanol, nonanol, decanol, undecanol, methyl acetate, ethyl acetate, butyl acetate, ethyl acetoacetate, diisopropyl adipate, and 2-(2-ethoxyethoxy)ethanol)(Transcutol®; and/or the C₂-C₃₀ diol is selected from the group consisting of propylene glycol, ethylene glycol, diethylene glycol, triethylene glycol, dipropylene glycol, propanediol, butanediol, pentanediol, polyethylene glycol, and dimethylisosorbate (DMI); and/or the C₃-C₃₀ polyol is selected from the group consisting of glycerol and hexanetriol; and/or the fatty alcohol is selected from the group consisting of oleyl alcohol, caprylic alcohol, decyl alcohol, lauryl alcohol, 2-lauryl alcohol, myristyl alcohol, cetyl alcohol, stearyl alcohol, linoleyl alcohol, and linolenyl alcohol; and/or the fatty acid or fatty acid ester is selected from the group consisting of octanoic acid, linoleic acid, valeric acid, heptanoic acid, pelagonic acid, caproic acid, capric acid, lauric acid, myristric acid, stearic acid, oleic acid, caprylic acid, isovaleric acid, neopentanoic acid, neoheptanoic acid, neonanoic acid, trimethyl hexaonic acid, neodecanoic acid, isostearic acid, ethyl oleate, isopropyl n-butyrate, isopropyl n-hexanoate, isopropyl n-decanoate, isopropyl myristate (IPM), isopropyl palmitate, octyldodecyl myristate, methyl valerate, methyl propionate, diethyl sebacate, ethyl oleate, butyl stearate, methyl laurate, glyceryl monolaurate, polypropyleneglycol octadecyl ether (Arlamol® E), and propylene glycol monolaurate; and/or the polyethoxylated fatty acid ester is polyoxyethylene (8) stearate (Myrj™ 45), or polyoxyethylene (40) stearate (Myrj™ 52); and/or the cyclic or N,N-dimethyl amide is selected from the group consisting of N,N-dimethylacetamide, N,N-dimethylformamide, N,N-dimethyloctamide, N,N-dimethyldecamide, 1-methyl-2-pyrrolidone, 2-pyrrolidone, 1-lauryl-2-pyrrolidone, 1-methyl-4-carboxy-2-pyrrolidone, 1-hexyl-4-carboxy-2-pyrrolidone, 1-lauryl-4-carboxy-2-pyrrolidone, 1-methyl-4-methoxycarbonyl-2-pyrrolidone, 1-hexyl-4-methoxycarbonyl-2-pyrrolidone, 1-lauryl-4-methoxycarbonyl-2-pyrrolidone, N-methyl-pyrrolidone (NMP), N-cyclohexylpyrrolidone, N-dimethylaminopropyl-pyrrolidone, N-cocoalkylpyrrolidones, N-tallowalkylpyrrolidones, C₁-C₂₀ esters of N-(2-hydroxyethyl)-2-pyrrolidone, and 1-dodecylazacycloheptane-2-one (Azone®); and/or the sorbitan monoester is selected from the group consisting of polysorbate 20, polysorbate 40, polysorbate 60, polysorbate 80, polysorbate 20, sorbitan laurate, sorbitan monopalmitate, sorbitan stearate, sorbitan tristearate, sorbitan monooleate, and sorbitan trioleate; and/or the polyethylene glycol ether is selected from the group selected from alkylaryl ethers of polyethylene glycol, polyethylene glycol monomethyl ethers, polyethylene glycol dimethyl ethers, and polyethylene glycol dodecyl ether (Brij® 30), polyethylene glycol oleyl ether (Brij® 93), polyoxyethylene 10 oleoyl ether (Brij® 97), and polyoxyethylene (20) oleyl ether (Brij® 99); and/or the biodegradable cyclic urea is a C₁-C₂₀ 1-alkyl-4-imidazolin-2-one; and/or the polysaccharide is 2-hydroxypropyl-β-cyclodextrin or 2,6-dimethyl-β-cyclodextrin; and/or the terpene or essential oil is selected from the group consisting of limonene, α-pinene, β-carene, α-terpineol, terpinen-4-ol, carvol, carvone, pulegone, piperitone, menthone, α-pinene oxide, cyclopentene oxide, 1,8-cineole, ylang ylang oil, anise oil, chenopodium oil, and eucalyptus oil; and/or the surfactant is selected from the group consisting of azelaic acid, poloxamer 231, poloxamer 182, poloxamer 184, sodium cholate, sodium salts of taurocholic acid, glycolic acid, desoxycholic acid, and poloxamer 407 (Synperonic® PE/F 127); and/or the sulfoxide is dimethylsulfoxide; and/or the fatty acid or polyoxyethylene triglyceride is selected from the group consisting of propylene glycol dicaprylate/dicaprate (Miglyol® 840), lecithin, and polyoxyethylene glyceride.

In certain embodiments, the penetration enhancer is selected from the group consisting of propylene glycol, NMP, polyoxypropylene (15) stearyl ether (Arlamol® E), DMI, 1-dodecylazacycloheptane-2-one (Azone®), sorbitan laurate, polysorbate 20, polysorbate 40, polysorbate 60, polysorbate 80, oleic acid, 2-(2-ethoxyethoxy)ethanol (Transcutol®), and IPM, or a combination thereof (e.g., a combination of oleic acid, NMP, IPM, and polysorbate 80).

In certain embodiments, the PSA is selected from the group consisting of an acrylic-based (e.g. an acylate copolymer), a rubber-based (e.g. a polyhydrocarbon copolymer), a silicone-based, a polyurethane-based, a polyester-based, and a polyether-based adhesive, or a combination thereof.

In certain embodiments, the PSA is an acrylate copolymer or a polyhydrocarbon copolymer.

In certain embodiments, the PSA is selected from the group consisting of acrylate-vinyl acetate copolymers, acrylate-2-ethylhexyl acrylate copolymers, acrylate-hydroxyethyl acrylate copolymers, acrylate-ethyl acrylate copolymers, acrylate-methyl methacrylate copolymers, acrylate-glycidyl methacrylate copolymers, polyisoprene copolymers, polybutylene copolymers, and polyisobutylene copolymers, or mixtures thereof.

In certain embodiments, the PSA is selected from the group consisting of Duro-Tak® 387-2516/87-2516, Duro-Tak® 87-2852, Duro-Tak® 387-2510/87-2510, Gelva® GMS 788, Duro-Tak® 87-9301, Duro-Tak® 87-202A, and Duro-Tak® 87-4098, or a combination thereof.

In certain embodiments, the transdermal drug composition comprises 2-(2-ethoxyethoxy)ethanol (Transcutol®) and oleic acid and at least one of the following: 1-dodecylazacycloheptane-2-one (Azone®), NMP, IPM, a polysorbate (Tween®), Eudagrit® RL100 or Eudagrit® L100, and/or a 60:40 random copolymer of vinyl pyrrolidone and vinyl acetate (Plasdone™ S-630); and a PSA such as Duro-Tak® 387-2516/87-2516.

In certain embodiments, the transdermal drug composition comprises 2-(2-ethoxyethoxy)ethanol (Transcutol®), oleic acid, NMP, IPM, a polysorbate (Tween®), and a PSA such as Duro-Tak® 387-2516/87-2516. In certain embodiments, the transdermal drug composition comprises 2-(2-ethoxyethoxy)ethanol (Transcutol®), oleic acid, NMP, IPM, a polysorbate (Tween®), Eudagrit® RL100 or Eudagrit® L100, and a PSA such as Duro-Tak® 387-2516/87-2516.

In certain embodiments, the opipramol salt is selected from the group consisting of opipramol tartrate, opipramol succinate, opipramol fumarate, opipramol mesylate, opipramol lactate, opipramol oleate, and opipramol azylate.

In certain embodiments, the opipramol is opipramol free base.

In certain embodiments, the opipramol is from about 1 to about 25%, or about 5% to about 20%, or about 7.5% to about 12.5% w/w based on the total weight of the composition. In certain embodiments, the opipramol is about 5% w/w based on the total weight of the composition. In certain embodiments, the opipramol is about 10% w/w based on the total weight of the composition. In certain embodiments, the opipramol is about 7.5% w/w based on the total weight of the composition. In certain embodiments, the opipramol is about 12.5% w/w based on the total weight of the composition. In certain embodiments, the opipramol is dissolved in the composition.

In certain embodiments, the transdermal drug composition comprises 0% to about 3% w/w hydrophilic polymer (e.g., Eudagrit® RL100 or Plasdone™); about 0.001% to about 30% w/w plasticizer (e.g., Transcutol®); about 5% to about 25% w/w of one or more penetration enhancers (e.g., one or more of oleic acid, NMP, IPM, Azone® and polysorbate 80); about 40% to about 80% w/w PSA; and about 1% to about 25% w/w opipramol or a pharmaceutically acceptable salt thereof.

In certain embodiments, the plasticizer is 2-(2-ethoxyethoxy)ethanol (Transcutol®) and the plasticizer comprises about 10% to about 30% w/w of the composition.

In certain embodiments, the penetration enhancer is oleic acid and the penetration enhancer comprises about 1% to about 10% w/w of the composition.

In certain embodiments, the hydrophilic polymer is a 60:40 random copolymer of vinyl pyrrolidone and vinyl acetate (Plasdone™ S-630) and the hydrophilic polymer comprises about 0.5% to about 3% w/w of the composition.

In certain embodiments:

the composition comprises about 1% to about 5% w/w of the penetration enhancer, wherein the penetration enhancer is 1-dodecylazacycloheptane-2-one (Azone®) and/or comprises about 0.1% to about 3% w/w of the plasticizer, wherein the plasticizer is polysorbate 80.

In certain embodiments:

the plasticizer is 2-(2-ethoxyethoxy)ethanol (Transcutol®) and comprises about 20% w/w of the composition; and/or the composition comprises at least one of the following penetration enhancers: up to about 5% w/w oleic acid, up to about 10% w/w polyoxypropylene (15) stearyl ether (Arlamol® E), up to about 10% w/w DMI, up to about 10% w/w IPM, and up to about 2% w/w polysorbate 80; and
the PSA comprises Duro-Tak® 387-2516/87-2516 and the PSA comprises about 53 to about 60% w/w of the composition.

In certain embodiments:

the plasticizer is 2-(2-ethoxyethoxy)ethanol (Transcutol®) and comprises about 20% w/w of the composition;
the penetration enhancer comprises about 1% to about 2% w/w polysorbate 80 and optionally comprises up to about 5% w/w NMP, up to about 5% w/w oleic acid, and up to about 5% w/w 1-dodecylazacycloheptane-2-one (Azone®);
the PSA is Duro-Tak® 387-2516/87-2516 and the PSA comprises about 41% to about 58% w/w of the composition; and
optionally further comprising up to about 2% w/w of a hydrophilic polymer, wherein the hydrophilic polymer is a 60:40 random copolymer of vinyl pyrrolidone and vinyl acetate (Plasdone™ S-630). In certain embodiments, the penetration enhancer comprises about 10% w/w IPM.

In certain embodiments, the plasticizer is 2-(2-ethoxyethoxy)ethanol (Transcutol®) and comprises about 20% w/w of the composition;

the penetration enhancer is a mixture of about 1% to about 5% w/w azelaic acid, about 10% w/w IPM, and about 0.2% to about 5% w/w polysorbate 80; and
the PSA is Duro-Tak® 387-2516/87-2516 and the PSA comprises about 49% to about 54.8% w/w of the composition.

In certain embodiments, the plasticizer is 2-(2-ethoxyethoxy)ethanol (Transcutol®) and comprises about 20% w/w of the composition;

the penetration enhancer is a mixture of about 5% w/w oleic acid, about 10% w/w IPM, and about 0.2% to about 5% w/w polysorbate 80; and
the PSA is Duro-Tak® 387-2516/87-2516 and the PSA comprises about 49% to about 54.8% w/w of the composition.

In certain embodiments, the composition further comprises about 0.5% to about 2% Eudragit® RL100 or Eudragit® L100. In certain embodiments, the composition further comprises about 1% w/w Eudragit® RL100.

In certain embodiments:

the plasticizer is 2-(2-ethoxyethoxy)ethanol (Transcutol®) and comprises about 20% w/w of the composition;
the penetration enhancer is a mixture of about 10% w/w IPM, and about 0.2% to about 5% w/w polysorbate 80; and
the PSA is Duro-Tak® 387-2516/87-2516 and the PSA comprises about 49% to about 54.8% w/w of the composition. In certain embodiments, the composition further comprises about 0.5% to about 2% Eudragit® RL100 or Eudragit® L100. In certain embodiments, the composition further comprises about 1% w/w Eudragit® RL100. In certain embodiments, the penetration enhancer comprises up to about 5% w/w oleic acid. In certain embodiments, the penetration enhancer comprises up to about 5% w/w azelaic acid.

In certain embodiments:

the plasticizer is 2-(2-ethoxyethoxy)ethanol (Transcutol®) and comprises about 20% w/w of the composition;
the penetration enhancer is a mixture of about 5% w/w oleic acid, up to about 5% w/w NMP, about 10% w/w IPM, and about 0.2% w/w polysorbate 80; and
the PSA comprises Duro-Tak® 387-2516/87-2516, and the PSA comprises about 53% to about 54.8% w/w of the composition. In certain embodiments, the composition further comprises about 0.5% to about 2% Eudragit® RL100 or Eudragit® L100. In certain embodiments, the composition further comprises about 1% w/w Eudragit® RL100.

In certain embodiments:

the plasticizer is 2-(2-ethoxyethoxy)ethanol (Transcutol®) and comprises about 20% w/w of the composition, or is oleyl alcohol and comprises about 10% w/w of the composition; the penetration enhancer optionally comprises up to about 5% w/w oleic acid and up to about 10% w/w IPM; and
the PSA comprises Duro-Tak® 387-2516/87-2516, and the PSA comprises about 59% to about 85% w/w of the composition. In certain embodiments, the composition further comprises about 0.5% to about 2% Eudragit® RL100 or Eudragit® L100. In certain embodiments, the composition further comprises about 1% w/w Eudragit® RL100. In certain embodiments, the penetration enhancer comprises about 1% to about 5% w/w oleic acid. In certain embodiments, the penetration enhancer comprises about 1% to about 10% w/w IPM.

In certain embodiments:

the plasticizer is 2-(2-ethoxyethoxy)ethanol (Transcutol®) and comprises about 5% to about 30% w/w of the composition;
the penetration enhancer is a mixture comprising about 5% to about 10% w/w oleic acid; about 2% w/w NMP; about 5% to about 10% w/w IPM; and about 1% w/w Tween® 80; and the PSA comprises Duro-Tak® 387-2516/87-2516, and the PSA comprises about 52% to about 77% w/w of the composition. In certain embodiments, the composition further comprises about 0.5% to about 2% Eudragit® RL100 or Eudragit® L100. In certain embodiments, the composition further comprises about 1% w/w Eudragit® RL100.

Furthermore, contemplated herein, in part, are transdermal compositions with two or more skin penetration enhancers, wherein the two or more skin penetration enhancers provide an additive or even a synergistic effect on the transdermal delivery of active agents. It is contemplated that the use of two or more disclosed skin penetration enhancers, each increasing skin permeability via a different mechanism, may be additive in their enhancing effects. In an embodiment, a disclosed combination of enhancers may even have a synergistic effect on skin penetration, i.e. an effect that is greater than the sum of the individual effects of the enhancers alone.

Pressure sensitive adhesives (PSA) and contact adhesives are available in a wide variety of chemical compositions or systems. Some of the most common types of systems contemplated for use in the present disclosure include, e.g., acrylic and methacrylate adhesives, rubber-based pressure sensitive adhesives, styrene copolymers (SIS/SBS), and silicones. Acrylic adhesives are known for excellent environmental resistance and fast-setting time when compared with other resin systems. Acrylic pressure sensitive adhesives often use an acrylate system. Ethylene ethyl acrylate (EEA) or ethylene methyl acrylate (EMA) copolymers are used to form hot melt PSA adhesives. Natural rubber, synthetic rubber or elastomer sealants and adhesives can be based on a variety of systems such silicone, polyurethane, chloroprene, butyl, polybutadiene, isoprene or neoprene. Rubber and elastomers are characterized by their high degree of flexibility and elasticity (high reversible elongation). Styrene-isoprene-styrene (SIS) and styrene-butadiene-styrene (SBS) copolymers are commonly applied in pressure sensitive adhesive applications. Silicone is produced through the hydrolysis and polymerization of silanes and siloxanes. In certain embodiments, the pressure-sensitive adhesive component of the disclosed compositions and patches is a polyacrylate adhesive, e.g. solution polymethacrylate. Such polyacrylates are made by copolymerizing one or more main acrylate monomers (“acrylate” is intended to include both acrylates and methacrylates), one or more modifying monomers, and one or more functional group-containing monomers in an organic solvent solution. The acrylate monomers used to make these polymers are normally alkyl acrylates of 4-17 carbon atoms, with 2-ethylhexylacrylate, butylacrylate and isooctylacrylate being preferred. Modifying monomers are typically included to alter the properties of the polymer such as tack. Examples of modifying monomers are acrylates such as ethyl acrylate, vinyl acetate, and methyl methacrylate. The functional group-containing monomer provides sites for crosslinking. In the polyacrylate of the present invention, the functional group(s) will normally be carboxyl, hydroxyl, or combinations thereof. Monomers that provide such groups are acids, e.g. acrylic acid, and hydroxy-containing monomers such as hydroxyethyl acrylate. Examples of such solution polyacrylates are disclosed in the art. See, for instance, U.S. Pat. No. 5,393,529, the disclosure of which with respect to such copolymers is incorporated herein. Preferred copolymers are those of 2-ethylhexylacrylate, vinyl acetate, hydroxyethyl acrylate, and glycidyl methacrylate. Preferred adhesive agents may comprise monomers such as vinyl acetate; 2-ethylhexyl acrylate; hydroxyethyl acrylate; and glycidyl methacrylate.

The term “pressure-sensitive adhesive” includes all polymers able to function as pressure-sensitive adhesives per se and those that function as a pressure-sensitive adhesive by admixture with tackifiers, plasticizers or other additives. The term “pressure-sensitive adhesive” also includes mixtures of different polymers and mixtures of polymers, such as polyisobutylenes (PIB), of different molecular weights, wherein each resultant mixture is a pressure-sensitive adhesive. Other useful rubber based pressure-sensitive adhesives include hydrocarbon polymers such as natural and synthetic polyisoprene, polybutylene and polyisobutylene, styrene/butadiene polymers styrene-isoprene-styrene block copolymers, hydrocarbon polymers such as butyl rubber, halogen-containing polymers such as polyacrylic-nitrile, polytetrafluoroethylene, polyvinylchloride, polyvinylidene chloride, and polychlorodiene, and other copolymers thereof.

Other useful PSAs can include acrylic-based and silicone-based PSAs such as those described in U.S. Pat. Nos. 5,474,783, and 5,656,386. Suitable commercially available acrylic-based polymers can include adhesives that are commercially available and include the polyacrylate adhesives sold under the trademarks Duro-Tak® by National Starch and Chemical Corporation, Bridgewater, N.J. Other suitable acrylic-based adhesives are those sold under the trademark Gelva-Multipolymer Solution® (GMS) (Monsanto; St. Louis, Mo.).

According to the present invention the acrylate adhesive may be crosslinked with sufficient aluminum acetylacetonate or other crosslinking agent to significantly improve the cohesive strength and cold flow properties of the adhesive relative to those of the uncrosslinked adhesive. The crosslinking density should be low since high degrees of crosslinking may adversely affect the tack and pull adhesion or yield a nontacky product. Normally the amount of aluminum acetylacetonate used is in the range of 0.1 to 1% by weight. In certain embodiments, the adhesive composition is crosslinked by mixing a solution of the polyacrylate, aluminum acetylacetonate, and drug in the desired proportions, causing the mixture to effect crosslinking, and then removing the solvent (e.g. by allowing to evaporate in air). Examples of solvents that may be used are ethyl acetate, ethanol, methanol, toluene, isopropyl alcohol and heptane. If desired, removal of the solvent by curing may be carried out at 40 to 1500° C. for 1 to 20 minutes. As indicated above, the adhesive compositions of the invention may be used to form the matrix (drug reservoir) component of a transdermal patch or be used as a separate in-line adhesive layer. In either application, the composition may define the basal surface (i.e. the surface that contacts the skin) of the patch when the patch is in use. As indicated, when the composition is used to form the matrix, the drug is incorporated into the adhesive before crosslinking. When the composition forms an in-line basal adhesive layer, the drug may be incorporated into the layer either before crosslinking or by equilibration after the patch has been assembled.

Contemplated transdermal compositions may further include a pharmaceutically acceptable excipient such as e.g., N-methylpyrrolidone, polyvinylpyrrolidone, propylene glycol, or polyethylene glycol, or a combination of one or more such excipients. For example, disclosed compositions may include polyols and esters thereof, such as propylene glycol, ethylene glycol, glycerol, butanediol, polyethylene glycol, polyethylene glycol monolaurate, and mixtures thereof. Contemplated compositions may additionally include one or more antioxidants or preservatives such as, for example, N-acetyl cysteine, sodium bisulfate, sodium metabisulfite, EDTA, glutathione, and ascorbic acid.

Disclosed transdermal compositions of the present disclosure may further include one or more surfactants. Suitable surfactants may include anionic surfactants, cationic surfactants, nonionic surfactants, bile salts, and lecithin. Examples of suitable anionic surfactants include sodium laurate, sodium lauryl sulfate, and sodium laureth sulfate. Suitable cationic surfactants include cetyltrimethyl ammonium bromide, tetradecyltrimethylammonium bromide, benzalkonium chloride, octadecyltrimethylammonium chloride, cethylpyridinium chloride, dodecyltrimethylammonium chloride, and hexadecyultrimethylammonium chloride. Examples of suitable nonionic surfactants include poloxamer 231, poloxamer 182, poloxamer 184, Brij® 30 (polyoxyethylene (4) lauryl ether), Brij® 93 (polyoxyethylene (2) oleyl ether), Brij® 96 (polyoxyethylene (20) oleyl ether), Brij® 99 (polyoxyl (10) oleyl ether), Span® 20 (sorbitan monolaurate), Span® 40 (sorbitane monopalmitate), Span® 60 (sorbitane monostearate), Span® 80 (sorbitane monooleate), Span® 85 (sorbitane trioleate), Tween® 20 (polyethylene glycol sorbitan monolaurate; polyoxyethylene (20) sorbitan monolaurate), Tween® 40 (polyoxyethylene (20) sorbitan monopalmitate), Tween® 60 (polyethylene glycol sorbitan monostearate; polyoxyethylene (20) sorbitan monostearate), Tween® 80 (polyethylene glycol sorbitan monooleate; polyoxyethylene (20) sorbitan monooleate), Myrj® 45 (polyoxyethylene (8) stearate), Myrj® 51 (polyoxyethylene stearate), Myrj® 52 (polyoxyethylene stearate), and Miglyol 840 (propylene glycol dicaprylate/dicaprat), among others. Examples of suitable bile salts include sodium cholate, and sodium salts of taurocholic, glycholic, and desoxycholic acids.

Disclosed transdermal compositions of the present disclosure may further include thickening agents including cellulose ethers such as hydroxypropyl methyl cellulose, hydroxypropyl cellulose, ethylcellulose, hydroxyethyl cellulose, and carboxymethyl cellulose. For example, in one embodiment, a transdermal composition may comprise about 0.1 to about 10 weight percent, for example, about 0.1 to about 9 weight percent, or about 0.1 to about 8 weight percent of cellulose ether such as hydroxypropyl methyl cellulose and/or hydroxypropyl cellulose, for example, Klucel® hydroxypropyl cellulose.

Disclosed transdermal compositions of the present disclosure may further include one or more carrier materials. Non-limiting examples of suitable carrier materials include water, emollients, sterols or sterol derivatives, natural and synthetic fats or oils, solidifying agents, viscosity enhancers, rheology enhancers, polyols, surfactants, alcohols, esters, silicones, clays, starch, cellulose, and other pharmaceutically acceptable carrier materials. As will be recognized by one skilled in the art, the relative amounts of components in the compositions of the disclosure that can be used to formulate the composition will be dictated by the nature of the composition. The levels can be determined by routine experimentation in view of the disclosure provided herein.

Disclosed transdermal compositions of the present disclosure may further include one or more emollients. Thus, in one embodiment, the composition of the disclosure can optionally include one or more emollient, which typically acts to soften, soothe, and otherwise lubricate and/or moisturize the skin. Suitable emollients that can be incorporated into the compositions include oils such as petrolatum based oils, petrolatum, vegetable based oils, mineral oils, natural or synthetic oils, alkyl dimethicones, alkyl methicones, alkyldimethicone copolyols, phenyl silicones, alkyl trimethylsilanes, dimethicone, dimethicone crosspolymers, cyclomethicone, lanolin and its derivatives, fatty acid esters, glycerol esters and derivatives, propylene glycol esters and derivatives, alkoxylated carboxylic acids, alkoxylated alcohols, fatty alcohols, and combinations thereof. The esters can be selected from cetyl palmitate, stearyl palmitate, cetyl stearate, isopropyl laurate, isopropyl myristate, isopropyl palmitate, and combinations thereof. The fatty alcohols include octyldodecanol, lauryl, myristyl, cetyl, stearyl, behenyl alcohol, and combinations thereof. Ethers such as eucalyptol, cetearyl glucoside, dimethyl isosorbic polyglyceryl-3 cetyl ether, polyglyceryl-3 decyltetradecanol, propylene glycol myristyl ether, and combinations thereof can also suitably be used as emollients. The composition may desirably include one or more emollient in an amount of from about 0.1% to about 95% by weight, more desirably from about 5% to about 75% by weight, and even more desirably from about 10% to about 50% by weight of the composition.

Disclosed transdermal compositions of the present disclosure may further include one or more stearol or stearol derivatives. Stearol and stearol derivatives which are suitable for use in the compositions of the present disclosure include, but are not limited to cholesterol, sitosterol, stigmasterol, ergosterol, C₁₀-C₃₀ cholesterol lanosterol esters, cholecalciferol, cholesteryl hydroxystearate, cholesteryl isostearate, cholesteryl stearate, 7-dehydrocholesterol, dihydrocholesterol, dihydrocholesteryl octyldecanoate, dihydrolanosterol, dihydrolanosteryl octyidecanoate, ergocalciferol, tall oil sterol, soy sterol acetate, lanasterol, soy sterol, avocado sterols, fatty alcohols, and combinations thereof. The composition of the invention can desirably include sterols, sterol derivatives or mixtures of both sterols and sterol derivatives in an amount of from about 0.1% to about 10% by weight, more desirably from about 0.5% to about 5% by weight, and even more desirably from about 0.8% to about 1% by weight of the composition.

Optionally, the composition may comprise a solidifying agent, which may function to solidify the composition so that the composition is a solid at room temperature, and may affect the hardness and melting point of the composition. The solidifying agent also provides a tackiness to the composition that improves the transfer to the skin of the wearer, such as when the composition is incorporated into a personal care product. Depending on the solidifying agent selected, the solidifying agent can also modify the mode of transfer so that the composition tends to fracture or flake off instead of actually rubbing off onto the skin of the wearer which can lead to improved transfer to the skin. The solidifying agent may further function as an emollient, occlusive agent, and/or moisturizer. The solidifying agents may include waxes as well as compounds that perform functionally as waxes.

The solidifying agents can be selected from alkyl siloxanes, polymers, hydrogenated vegetable oils having a melting point of 35° C. or greater and fatty acid esters with a melting point of 35° C. or greater. Additionally, the solidifying agents can be selected from animal, vegetable and mineral waxes and alkyl silicones. Examples of solidifying agents include, but are not limited to, alkyl trimethylsilanes, beeswax, C₂₄-C₂₈ alkyl dimethicone, C₃₀ alkyl dimethicone, cetyl methicone, stearyl methicone, cetyl dimethicone, stearyl dimethicone, cerotyl dimethicone, candelilla wax, carnauba, cerasin, hydrogenated microcrystalline wax, jojoba wax, microcrystalline wax, lanolin wax, ozokerite, paraffin, spermaceti wax, cetyl esters, behenyl behenate, C₂₀-C₄₀ alkyl behenate, C₁₂-C₁₅ lactate, cetyl palmitate, stearyl palmitate, isosteryl behenate, lauryl behenate, stearyl benzoate, behenyl isostearate, cetyl myristate, cetyl octanote, cetyl oleate, cetyl ricinoleate, cetyl stearate, decyl oleate, diC₁₂-C₁₅ alkyl fumerate, dibehenyl fumerate, myristyl lactate, myristyl lignocerate, myristyl myristate, myristyl stearate, lauryl stearate, octyldodecyl stearate, octyldodecyl stearoyl stearate, oleyl arachidate, oleyl stearate, tridecyl behenate, tridecyl stearate, tridecyl stearoyl stearate, pentaerythrityl tetrabehenate, pentaerythrityl hydrogenated rosinate, pentaerythrityl distearate, pentaerythrityl tetraabeite, pentaerythrityl tetracocoate, pentaerythrityl tetraperlargonate, pentaerythrityl tetrastearate, theylene vinyl acetate, polyethylene, hydrogenated vegetable oil, hydrogenated squalene, squalene, hydrogenated coconut oil, hydrogenated jojoba oil, hydrogenated palm oil, hydrogenated palm kernel oil, hydrogenated olive oil, polyamides, metal stearates and other metal soaps, C₃₀-C₆₀ fatty alcohols, C₂₀₊ fatty acids, polypropylene, polystyrene, polybutane, polybutylene terephthalate, polydipentane, zinc stearate, and combinations thereof. The composition may desirably include one or more solidifying agents in an amount of from about 0.1% to about 95% by weight, more desirably from about 5% to about 75% by weight, and even more desirably from about 10% to about 50% by weight of the composition.

Optionally, one or more viscosity enhancers may be added to the composition to increase the viscosity, to help stabilize the composition, such as when the composition is incorporated into a personal care product, thereby reducing migration of the composition and improve transfer to the skin. Suitable viscosity enhancers include polyolefin resins, lipophilic/oil thickeners, ethylene/vinyl acetate copolymers, polyethylene, silica, silica silylate, silica methyl silylate, colloidal silicone dioxide, cetyl hydroxy ethyl cellulose, other organically modified celluloses, PVP/decane copolymer, PVM/MA decadiene crosspolymer, PVP/eicosene copolymer, PVP/hexadecane copolymer, clays, carbomers, acrylic based thickeners, and combinations thereof. The composition may desirably include one or more viscosity enhancers in an amount of from about 0.1% to about 25% by weight, more desirably from about 0.5% to about 20% by weight, and even more desirably from about 1% to about 10% by weight of the composition.

The compositions of the disclosure may optionally further comprise rheology enhancers. Rheology enhancers may help increase the melt point viscosity of the composition so that the composition readily remains on the surface of a personal care product and does not substantially migrate into the interior of the product, while substantially not affecting the transfer of the composition to the skin. Additionally, the rheology enhancers help the composition to maintain a high viscosity at elevated temperatures, such as those encountered during storage and transportation.

Suitable rheology enhancers include combinations of alpha-olefins and styrene alone or in combination with mineral oil or petrolatum, combinations of di-functional alpha-olefins and styrene alone or in combination with mineral oil or petrolatum, combinations of alpha-olefins and isobutene alone or in combination with mineral oil or petrolatum, ethylene/propylene/styrene copolymers alone or in combination with mineral oil or petrolatum, butylene/ethylene/styrene copolymers alone or in combination with mineral oil or petrolatum, ethylene/vinyl acetate copolymers, polyethylene polyisobutylenes, polyisobutenes, polyisobutylene, dextrin palmitate, dextrin palmitate ethylhexanoate, stearoyl inulin, stearalkonium bentonite, distearadimonium hectorite, and stearalkonium hectorite, styrene/butadiene/styrene copolymers, styrene/isoprene/styrene copolymers, styrene-ethylene/butylene-styrene copolymers, styrene-ethylene/propylene-styrene copolymers, (styrene-butadiene) n polymers, (styrene-isoprene) n polymers, styrene-butadiene copolymers, and styrene-ethylene/propylene copolymers and combinations thereof. Specifically, rheology enhancers such as mineral oil and ethylene/propylene/styrene copolymers, and mineral oil and butylene/ethylene/styrene copolymers (Versagel blends from Penreco) are particularly preferred. Also, Vistanex (Exxon) and Presperse (Amoco) polymers are particularly suitable rheology enhancers. The composition of the invention can suitably include one or more rheology enhancer in an amount of from about 0.5% to about 5% percent by weight of the composition.

In certain embodiments, the compositions may optionally comprise water. In these embodiments, the compositions can suitably comprise water in an amount of from about 0.1% (by weight of the composition) to about 99% (by weight of the composition), more preferably from about 10% (by weight of the composition) to about 90% (by weight of the composition), and still more preferably from about 30% (by weight of the composition) to about 85% (by weight of the composition).

Further components that may be included in transdermal compositions are disclosed in U.S. Pat. No. 7,879,344, the contents of which are hereby incorporated by reference.

A disclosed transdermal composition may have a physiologically acceptable pH. The term “physiologically acceptable pH” is understood to mean a pH that facilitates administration of the composition to a patient without significant adverse effects, e.g. a pH of about 4 to about 10.

Also provided herein are transdermal compositions that allow for enhanced delivery of active agents over an extended period of time. For example, a contemplated transdermal composition comprising a penetration enhancer and hydrophilic polymer may deliver more than two times, three times, or more of the active agent than compositions that do not include such penetration enhancer and hydrophilic polymer.

Specific combinations of components contemplated herein are shown in Tables A-1 to A-4 below.

TABLE A-1

| | 1 | 2 | 3 | 4 |
|---|---|---|---|---|
| Active Agent (e.g., opipramol | 1-50% | 1-35% | 1-25% | 2.5-25% opipramol or |
| or a pharmaceutically | | | | pharmaceutically |
| acceptable salt thereof | | | | acceptable salt thereof |
| C₈-C₃₀ fatty acid | 1-25% | 2.5-25% | 2.5-20% | 2-10% |
| (e.g. oleic acid) | | | | |
| C₁-C₁₀ monoalkylated C₄-C₁₆ | 2.5-40% | 3.5-30% | 5-30% | 5-25% |
| polyethylene glycol (e.g. | | | | |
| Transcutol ®) | | | | |
| Cyclic amide | Not | Not | Not | Not |
| | present; or | present; or | present; or | present; or |
| | up to 10% | up to 8% | up to 5% | up to 4% |
| C₈-C₃₀ fatty acid ester (e.g. | Not | Not | Not | Not |
| isopropyl myristate) | present; or | present; or | present; or | present; or |
| | up to 30% | up to 30% | up to 20% | up to 20% |
| Polysorbate or sorbitan ester | Not | Not | Not | Not |
| (e.g., polysorbate 80) | present; or | present; or | present; or | present; or |
| | up to 10% | up to 4% | up to 3% | up to 2% |
| PSA (dry) | 30-85% | 35-80% | 40-75% | 40-65% |

TABLE A-2

| | 1 | 2 | 3 | 4 |
|---|---|---|---|---|
| Opipramol or pharmaceutically | 1-40% | 1-25% | 1-25% | 2.5-25% |
| acceptable salt thereof | | | | |
| C₁₀-C₂₆ fatty acid | 2-25% | 2.5-20% | 2.5-15% | 2.5-10% |
| C₁-C₈ monoalkylated C₄-C₁₀ | 1-40% | 2-30% | 5-30% | 5-25% |
| polyethylene glycol (e.g. Transcutol ®) | | | | |
| N-C₁-C₆ alkylpyrrolidone and/or 1- | Not | Not | Not | Not |
| dodecylazacycloheptane-2-one | present; or | present; or | present; or | present; or |
| (Azone ®) | up to 10% | up to 8% | up to 5% | up to 4% |
| C₁₀-C₁₈ fatty acid C₁-C₈ alkyl ester | Not | 1-25% | 2-25% | 5-20% |
| (e.g. isopropyl myristate) | present; or | | | |
| | up to 30% | | | |
| Polysorbate or sorbitan ester | Not | Not | Not | 0.1-3% |
| | present; or | present; or | present; or | |
| | up to 7% | up to 4% | up to 3% | |
| PSA (dry) | 30-90% | 35-85% | 40-80% | 40-75% |

In certain embodiments of the compositions described in Table A-2, a polymethacrylate polymer, a polyvinylpyrrolidone polymer, or a combination thereof (e.g., poly(methacrylic acid-co-methyl methacrylate) 1:1 (Eudragit® L100) and/or copolymer of vinyl pyrrolidone and vinyl acetate (e.g. Plasdone™ S-630)) is present in the amount of 0.01-5%, 0.01-4%, 0.1-3%, 0.5-3% or 0.1-2.5% w/w.

TABLE A-3

| | 1 | 2 | 3 | 4 |
|---|---|---|---|---|
| Opipramol or pharmaceutically | 1-50% | 1-35% | 1-25% | 2.5-25% |
| acceptable salt thereof | | | | |
| C₁₆-C₂₀ mono- or di-unsaturated | 1-25% | 1-25% | 2-15% | 2-10% |
| fatty acid (e.g. oleic acid) | | | | |
| C₁-C₆ monoalkylated C₄-C₈ | 1-40% | 2-30% | 5-30% | 5-25% |
| polyethylene glycol (e.g. | | | | |
| Transcutol ®) | | | | |
| N-C₁-C₆ alkylpyrrolidone and/or 1- | 0.1-10% | 0.5-8% | 1-5% | 1-5% |
| dodecylazacycloheptane-2-one | | | | |
| (Azone ®) | | | | |
| C₁₀-C₁₈ fatty acid C₂-C₆ alkyl ester | 1-30% | 1-25% | 2-25% | 5-25% |
| (e.g. isopropyl myristate) | | | | |
| Polysorbate 80 (Tween ® 80) | 0.01-7% | 0.01-4% | 0.01-3% | 0.1-3% |
| poly(methacrylic acid-co-methyl | Not | 0.01-4% | 0.1-3% | 0.1-2.5% |
| methacrylate) 1:1 (Eudragit ® | present; or | | | |
| RL100 or L100) and/or | 0.01-5% | | | |
| copolymer of vinyl pyrrolidone | | | | |
| and vinyl acetate (e.g. Plasdone ™ | | | | |
| S-630) | | | | |
| PSA (dry) | 40-75% | 40-75% | 40-70% | 40-65% |

TABLE A-4

| | 1 | 2 | 3 | 4 | 5 |
|---|---|---|---|---|---|
| Opipramol base | 2.5-20% | 2.5-20% | 2.5-20% | 2.5-20% | 2.5-20% |
| Oleic acid | 2.5-7.5% | 2.5-7.5% | 2.5-7.5% | 2.5-7.5% | 2.5-7.5% |
| Transcutol ® | 10-20% | 10-20% | 10-20% | 10-20% | 10-20% |
| N-methyl alkylpyrrolidone | 1-5% | 1-5% | 1-5% | 1-5% | 1-5% |
| (NMP) | | | | | |
| 1-dodecylazacycloheptane-2- | Not | Not | Not | Not | Not |
| one (Azone ®) | present | present; | present | present; | present; |
| | | or 1-3% | | or 1-3% | or 1-3% |
| Isopropyl myristate (IPM) | 8-12% | 8-12% | 8-12% | 8-12% | 8-12% |
| Polysorbate 80 (Tween ® 80) | 0.2-3% | 0.2-3% | 0.2-3% | 0.2-3% | 0.2-3% |
| poly(methacrylic acid-co- | Not | Not | 0.1-3% | 0.1-2.5% | Not |
| methyl methacrylate) 1:1 | present | present | | | present |
| (Eudragit ® RL100 or L100) | | | | | |
| 60:40 linear random | 1-3% | 1-3% | Not | Not | Not |
| copolymer of vinyl | | | present | present | present |
| pyrrolidone and vinyl acetate | | | | | |
| (Plasdone ™ S-630) | | | | | |
| PSA (dry) | 40-65% | 40-65% | 40-65% | 40-65% | 45-60% |

Thus, as shown in the Tables above, the active agent (or opipramol base or pharmaceutically acceptable salt thereof), may represent, e.g., 1-50%, 1-35%, 1-25%, 2.5-25% 1-40%, 1-35%, or 2.5-20% w/w of the composition. The C₈-C₃₀, C₁₀-C₂₆, or C₁₆-C₂₀ mono- or di-unsaturated fatty acid (e.g. oleic acid) may represent, e.g., 1-25%, 2.5-25%, 2.5-20%, 2-10%, 2.5-7.5%, or 2-15%, of the composition. The C₁-C₁₀ monoalkylated C₄-C₁₆ polyethylene glycol (e.g. Transcutol®), C₁-C₈ monoalkylated C₄-C₁₀ polyethylene glycol (e.g. Transcutol®), C₁-C₆ monoalkylated C₄-C₈ polyethylene glycol (e.g. Transcutol®), or Transcutol® may represent, e.g., 1-40%, 5-30%, 5-25%, 2-30%, 2.5-40%, or 3.5-30%, of the composition. The cyclic amide, N—C₁-C₆ alkylpyrrolidone and/or 1-dodecylazacycloheptane-2-one (Azone®), or N-methylpyrrolidone (NMP), may each be not present, or may each be present in e.g., 1-3%, 0.1-10%, 0.5-8%, 1-5%, up to 10%, up to 8%, up to 5%, or up to 4%, of the composition. The C₈-C₃₀ fatty acid ester, C₁₀-C₁₈ fatty acid C₁-C₈ alkyl ester, C₁₀-C₁₈ fatty acid C₂-C₆ alkyl ester (e.g., isopropyl myristate), or isopropyl myristate (IPM), when present, may be present in, e.g., 8-12%, 1-30%, 1-25%, 2-25%, 5-25%, up to 30%, 5-20%, or up to 20%, of the composition. The polysorbate or sorbitan ester (e.g., polysorbate 80), or polysorbate 80 (Tween® 80), when present, may be present in, e.g., 0.2-3%, 0.01-7%, 0.01-4%, 0.01-3%, 0.1-3%, up to 7%, up to 4%, up to 3%, up to 10%, up to 4%, or up to 2%, of the composition. The PSA is present in, e.g., 30-85%, 35-80%, 40-75%, 40-65%, 30-90%, 35-85%, 40-80%, 40-70%, or 45-60%, of the composition.

### Active Agents

Provided herein are pharmaceutically acceptable transdermal compositions that include one or more active agents. In certain embodiments, the active agent is a dibenzazepine (iminostilbene) based active agent such as a dibenzazepine-based tricyclic antidepressant, for example a tricyclic antidepressant such as clomipramine, desipramine, doxepin, imipramine, imipraminoxide, lofepramine, metapramine, opipramol, quinupramine, and trimipramine. In certain embodiments, the active agent is opipramol.

Also contemplated are pharmaceutically acceptable salts of the disclosed active agents, e.g. opipramol. Pharmaceutically acceptable salts of the disclosed therapeutic or active agent can be synthesized by conventional chemical methods. Generally, such salts can be prepared by reacting the free acid or base forms of the agents with a stoichiometric amount of the appropriate base or acid in water or in an organic solvent, or in a mixture of the two; generally, non-aqueous media like propylene glycol, ether, ethyl acetate, ethanol, isopropanol, or acetonitrile. Lists of suitable salts are found in Remington's Pharmaceutical Sciences, 20th ed., Lippincott Williams & Wilkins, Baltimore, Md., 2000, p. 704.

Active agents may be present in the disclosed compositions in varying amounts, e.g. a disclosed composition may include for example about 1 to about 25 weight percent opipramol, about 1 to about 20 weight percent opipramol, about 5 to 20 weight percent, about 7.5 to about 12.5 weight percent, e.g., about 2, 3, 4, 5, 6, 9, 10, 11, 12, 13, 14, or 15 weight percent opipramol.

Specific contemplated pharmaceutically acceptable salts of opipramol include, but are not limited to, opipramol tartrate, opipramol succinate, opipramol fumarate, opipramol mesylate, opipramol lactate, opipramol oleate, and opipramol azylate.

### Transdermal Patches

In one aspect, the present disclosure relates to a transdermal delivery device for the transdermal delivery of opipramol comprising:
a. an inert layer detachable when used;
b. at least one adhesive layer comprising a transdermal drug composition as described above, wherein the adhesive layer is directly affixed to a surface of the inert layer; and
c. a backing layer, coated over the adhesive layer.

In certain embodiments, the device comprises two to ten adhesive layers, wherein each adhesive layer is coated on top of an adjacent adhesive layer.

In certain embodiments, the device comprises two to five adhesive layers.

In certain embodiments, the first adhesive layer is fixed to the inert layer and last adhesive layer is coated over the backing layer.

In certain embodiments, the thickness of each adhesive layer is in a range of about 0.08 mm to about 0.4 mm. In certain embodiments, said thickness is measured in a dry state. In certain embodiments, once the inert layer is removed the adhesive layer is placed on the skin of a patient.

In certain embodiments, the adhesive layer of the device comprises by weight:

about 10% opipramol base;
about 5% oleic acid;
about 20% 2-(2-ethoxyethoxy)ethanol (Transcutol®);
about 2% NMP;
about 10% IPM;
about 2% 1-dodecylazacycloheptane-2-one (Azone®);
about 2% polysorbate 80; and
about 49% PSA.

A transdermal device, e.g. patch, described herein may contain a number of elements. The backing layer, which may be adhered to the drug reservoir layer, serves as the upper layer of the patch during use, and functions as the primary structural element of the patch. The backing layer is made of a sheet or film of a preferably flexible elastomeric material that is substantially impermeable to the opipramol composition. The thickness of the layer is not particularly limited and can be appropriately chosen depending on the application, but will typically be on the order of 1.0 to about 4.0 millimeters in thickness. Preferably, the backing layer is composed of a material that permits the patch to follow the contours of the skin, such that it may be worn comfortably on any skin area, e.g., at joints or other points of flexure. In this way, in response to normal mechanical strain, there is little or no likelihood of the patch disengaging from the skin due to differences in the flexibility or resiliency of the skin and the patch. Examples of polymers useful for the backing layer include polyethylene, polypropylene, polyesters, polyurethanes, polyvinyl chloride, polyethylene vinyl acetate, polyvinylidene chloride, block copolymers, nylon, an unwoven fabric, and the like. The backing layer may also comprise laminates of one or more of the foregoing polymers.

The drug reservoir layer typically comprises a contact adhesive which is a pressure-sensitive adhesive suitable for long-term skin contact. The adhesive is preferably also physically and chemically compatible with the opipramol and with any carriers or vehicles incorporated into an opipramol composition. Further, the adhesive selected for use in the reservoir layer is preferably such that the opipramol is at least somewhat soluble in the adhesive. The thickness of the drug reservoir layer is not particularly limited, but will generally be in the range of about 0.2 to about 4 millimeters in thickness. Suitable adhesives for use in the drug reservoir include polysiloxanes, polyacrylates, polyurethanes, tacky rubbers such as polyisobutylene, and the like. Particularly preferred contact adhesives for use in the drug reservoir herein are cross-linked acrylates.

Optionally, the patch may further comprise a release liner. The release liner is a disposable element which serves to protect the patch prior to application. Typically, the release liner is formed from a material impermeable to the opipramol, any carriers or vehicles, and adhesive, and is easily stripped from the contact adhesive that serves as part of the drug reservoir layer. Preferred release liners for use herein are those which are generally suitable for use in conjunction with pressure-sensitive adhesives, such as silanized polyester films, among others.

In certain embodiments the compositions and devices (e.g. transdermal patches) comprising such compositions of the present disclosure provide opipramol at a pre-determined delivery rate to a patient, for example wherein the pre-determined delivery rate is substantially continuous over at least 12 hours, or over at least 1 day, or over at least 3 days, or over at least 7 days (one week).

In some embodiments, the daily dosage may include about 5 mg/day to about 60 mg/day, or about 10 mg/day to about 40 mg/day of opipramol. In this way, opipramol may be effective for at least about 1 day, or at least about 3 days or more.

Opipramol may be, for example, administered at a dosage of about 5 mg or about 60 mg/day, e.g. about 10 mg/day to about 40 mg/day, about 15 mg/day to about 30 mg/day, about 30 mg/day to about 70 mg/day or about 40 mg/day to about 60 mg/day. For example, 40 mg/day, or 30 mg/day, 20 mg/day or 15 mg/day of opipramol may be administered. In another embodiment, opipramol may be administered from about 0.2 mg/kg/day to about 1.5 mg/kg/day.

In some embodiments, compositions contemplated herein may be a gel, gel-like, or liquid at room temperature.

### Methods of Treatment

The present disclosure also relates to the use of a disclosed composition or transdermal drug delivery device in the treatment of a disease or condition.

In one aspect, the present disclosure relates to a method of treating a patient having a disorder selected from the group consisting of central nervous system (CNS) disorders, peripheral nervous system disorders, factitious disorders, somatoform disorders, inflammatory disorders, and pain-related disorders, said method comprising the steps of:
a. providing a transdermal delivery device as described above; and
b. placing an adhesive layer of the device against the skin of the patient, thereby providing an amount of opipramol effective to treat the disorder.

In one aspect, the present disclosure relates to a method of preventing, treating or suppressing tobacco or nicotine dependence or usage in a patient, said method comprising the steps of:
a. providing a transdermal delivery device as described above;
b. placing the adhesive layer of the device against the skin of the patient, thereby providing an amount of opipramol effective to prevent, treat, or suppress the tobacco or nicotine dependence or usage.

In certain embodiments, the adhesive layer is placed on the skin of the patient for a period of 1 to 10 days.

In certain embodiments, the therapeutically effective amount of opipramol is about 5 mg/day to about 60 mg/day.

In certain embodiments, said CNS disorder is selected from the group consisting of epilepsy, Parkinson's disease, Alzheimer's disease, depression, restless legs syndrome, pain, schizophrenia, neurodegeneration, dementia, and migraine.

In certain embodiments, said inflammatory disorder is selected from the group consisting of rheumatoid arthritis, osteoarthritis, ankylosing spondylitis, tendinitis, bursitis, and acute gout.

In certain embodiments, said somatoform disorder is selected from the group consisting of anxiety, phobias, generalized anxiety disorder, social anxiety disorder, panic disorder, agoraphobia, obsessive-compulsive disorder, post-traumatic stress disorder, bipolar disorder, attention deficit hyperactivity disorder, a sleep disorder, and a cognitive disorder.

In one aspect, the present disclosure relates to the use of a transdermal drug composition as described above in the manufacture of a medicament for treatment of any disease or condition for which opipramol provides a therapeutic benefit.

The present disclosure now being generally described, it will be more readily understood by reference to the following Examples, which are included merely for purposes of illustration of certain aspects and embodiments of the present disclosure, and are not intended to limit the present disclosure in any way.

### Examples

### Patches—General Procedures

Opipramol base was mixed with solubilizer and different enhancers. Suitable adhesive elements were added to the mixture. The mixture was stirred to obtain homogenous viscous solutions. The solutions were casted on siliconized release liner, using a laboratory-casting knife to form 0.4 mm membranes. The membranes were dried for 30 min at 70° C. to remove the solvents to create a dry film. The dried films were laminated on polyester/EVA backing in the required dimensions. Another layer of dry film was applied on top of the first layer. This procedure produced a dry patch composite of two layers. A multilayer patch can be prepared in a similar way by applying a number of dry films one on top of the other.

### Example 1

The effects of different concentrations of opipramol on the transdermal delivery of opipramol through full thickness pig skin were evaluated using the Franz Cell delivery system. Dry patches containing opipramol, oleic acid, Transcutol®, Azone®, NMP, IPM, Tween® 80 and PSA were prepared (formulations 1-6). Samples were collected from the receiver cell at 22, 29 and 42 hours after application of the patch formulation to the skin. The amount of opipramol compounds in the receiver cell fluid was determined using a spectrophotometer at 280 nm.

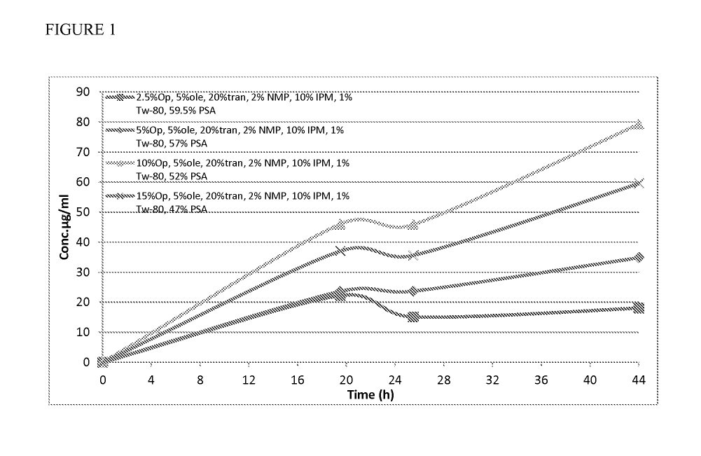

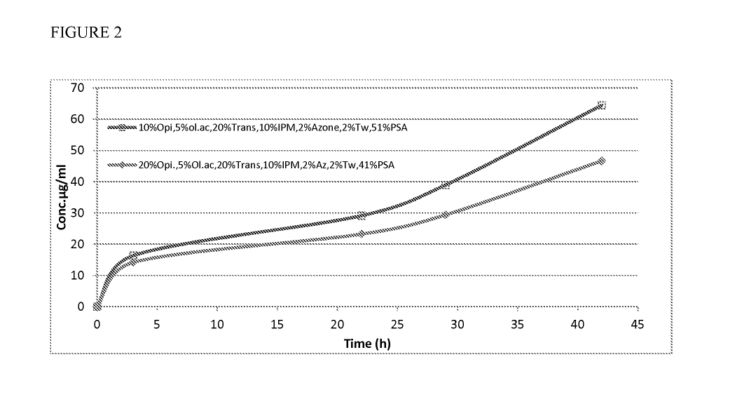

FIGS. 1 and 2, and Table 1 (corresponding to FIG. 1) and Table 2 (corresponding to FIG. 2) tabulate the influence of different opipramol concentrations on the transdermal delivery of opipramol through pig skin, ex vivo.

TABLE 1

| | 1 | 2 | 3 | 4 |
|---|---|---|---|---|
| Opipramol base % | 2.5 | 5 | 10 | 15 |
| Oleic acid % | 5 | 5 | 5 | 5 |
| Transcutol ® % | 20 | 20 | 20 | 20 |
| NMP % | 2 | 2 | 2 | 2 |
| IPM % | 10 | 10 | 10 | 10 |
| Tween ® 80% | 1 | 1 | 1 | 1 |
| PSA (dry) % | 59.5 | 57 | 52 | 47 |
| Conc. μg/mL at 44 hr | 20 | 35 | 80 | 60 |

TABLE 2

| | 5 | 6 |
|---|---|---|
| Opipramol base % | 10 | 20 |
| Oleic acid % | 5 | 5 |
| Transcutol ® % | 20 | 20 |
| IPM % | 10 | 10 |
| Azone ® % | 2 | 2 |
| Tween ® 80% | 2 | 2 |
| PSA (dry) % | 51 | 41 |
| Conc. μg/mL at 42 hr | 65 | 47.5 |

From FIGS. 1 and 2, patches comprising 10% opipramol show an improved transdermal delivery of opipramol in comparison with patches comprising 2.5, 5, 15, or 20% opipramol. Furthermore, patches including 1% Tween® 80 and 2% NMP have significantly improved transdermal delivery of opipramol over patches comprising 2% Tween® 80 and 2% Azone®.

### Example 2

The transdermal delivery of opipramol through full thickness pig skin was evaluated using the Franz Cell delivery system. Dry patches containing opipramol, oleic acid, oleyl alcohol, Azelaic acid, Transcutol®, IPM, Tween® 80, Eudragit® L100 and PSA were prepared (formulations 7-18). Samples were collected from the receiver cells at 27 hours at 29 and 41 hours after application of the formulation to the skin. The amount of opipramol in the receiver cell fluid was determined using a spectrophotometer at 280 nm.

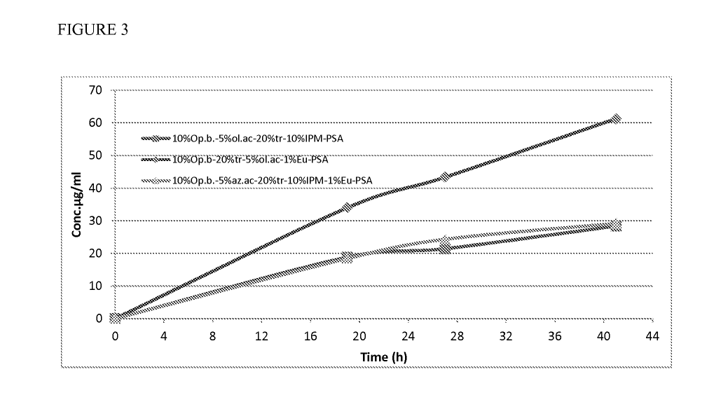

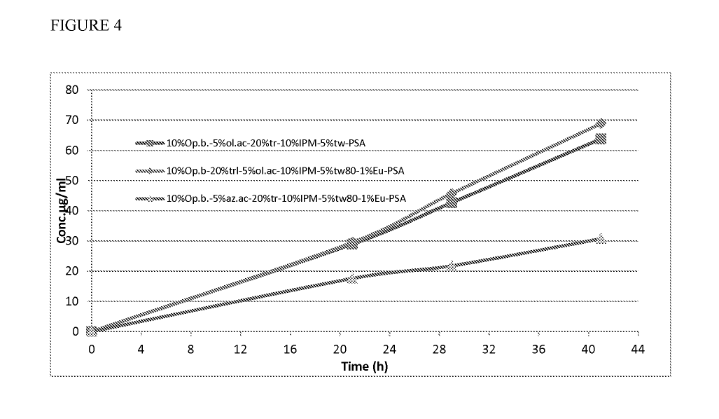

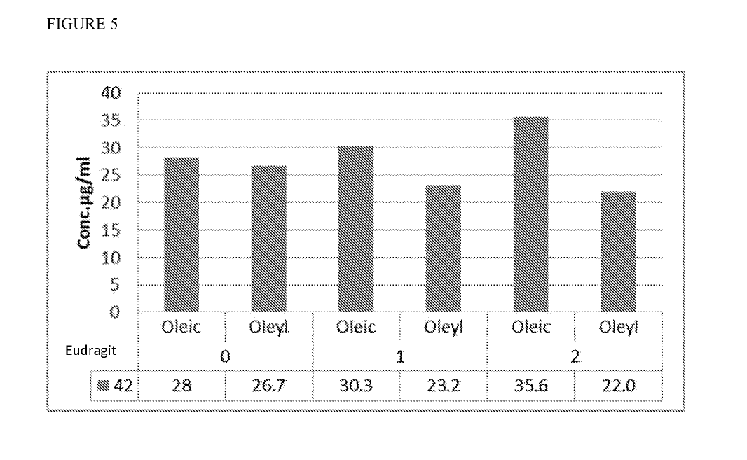

FIGS. 3, 4, and 5, and Table 3 (corresponding to FIG. 3), Table 4 (corresponding to FIG. 4), and Table 5 (corresponding to FIG. 5) indicate that patches containing 5% oleic acid showing higher transdermal delivery of opipramol through pig skin, ex vivo in comparison to patches containing 5% azelaic acid or 10% oleyl alcohol.

TABLE 3

| | 7 | 8 | 9 |
|---|---|---|---|
| Opipramol base % | 10 | 10 | 10 |
| Transcutol ® % | 20 | 20 | 20 |
| Oleic acid % | 5 | 5 | |
| Azelaic acid % | | | 5 |
| IPM % | 10 | 10 | 10 |
| Tween ® 80% | 0.2 | 0.2 | 0.2 |
| Eudragit L100% | | 1 | 1 |
| PSA (dry) % | 54.8 | 53 | 53 |
| Conc. μg/mL at 41 hr | 30 | 62 | 30 |

TABLE 4

| | 10 | 11 | 12 |
|---|---|---|---|
| Opipramol base % | 10 | 10 | 10 |
| Transcutol ® % | 20 | 20 | 20 |
| Oleic acid % | 5 | 5 | |
| Azelaic acid % | | | 5 |
| IPM % | 10 | 10 | 10 |
| Tween ® 80% | 5 | 5 | 5 |
| Eudragit ® L100% | | 1 | 1 |
| PSA (dry) % | 50 | 49 | 49 |
| Conc. μg/mL at 41 hr | 63 | 70 | 30 |

TABLE 5

| | 13 | 14 | 15 | 16 | 17 | 18 |
|---|---|---|---|---|---|---|
| Opipramol base % | 5 | 5 | 5 | 5 | 5 | 5 |
| Oleic acid % | 4 | | 4 | | 4 | |
| Oleyl alcohol % | | 10 | | 10 | | 10 |
| Transcutol ® % | 20 | | 20 | | 20 | |
| IPM % | 10 | | 10 | | 10 | |
| Eudragit ® L100% | | | 1 | 1 | 2 | 2 |
| PSA (dry) % | 61 | 85 | 60 | 84 | 59 | 83 |
| Conc. μg/mL at 42 hr | 28 | 27 | 30 | 23 | 36 | 22 |

FIGS. 3 and 4 demonstrate that patches containing 5% Tween® 80 with or without Eudragit® L100 show no significant difference in the transdermal delivery of opipramol in comparison with patches containing 0.2% Tween® 80. Patches comprising a combination of Eudragit® and oleic acid show significantly better transdermal delivery of opipramol in comparison with patches comprising Eudragit® and oleyl alcohol or patches containing a combination of Eudragit® and azelaic acid. Tween® 80 has no impact on the transdermal delivery of opipramol when Eudragit® is absent from the patch (FIG. 4).

### Example 3

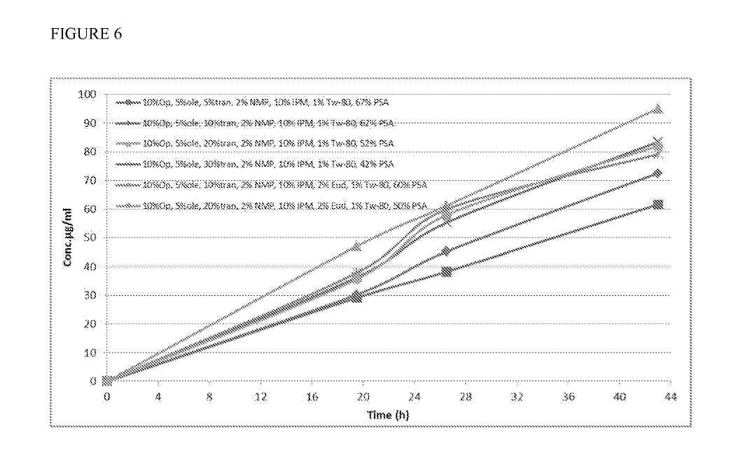

FIG. 6 depicts the effect of Transcutol® on the transdermal delivery of opipramol. The effects of different concentrations of Transcutol® on the transdermal delivery of opipramol through full thickness pig skin were evaluated using the Franz Cell delivery system. Dry patches containing opipramol, oleic acid, Transcutol®, Eudragit® RL100, NMP, IPM, Tween® 80 and PSA were prepared (formulations 19-24). Samples were collected from the receiver cell at 19, 26, and 43 hours after application of the formulation to the skin. The amount of opipramol compounds in the receiver cell fluid was determined using a spectrophotometer at 280 nm.

TABLE 6

| | 19 | 20 | 21 | 22 | 23 | 24 |
|---|---|---|---|---|---|---|
| Opipramol base % | 10 | 10 | 10 | 10 | 10 | 10 |
| Oleic acid % | 5 | 5 | 5 | 5 | 5 | 5 |
| Transcutol ® % | 5 | 10 | 20 | 30 | 10 | 20 |
| NMP % | 2 | 2 | 2 | 2 | 2 | 2 |
| IPM % | 10 | 10 | 10 | 10 | 10 | 10 |
| Eudragit ® RL100% | | | | | 2 | 2 |
| Tween ® 80% | 1 | 1 | 1 | 1 | 1 | 1 |
| PSA (dry) % | 67 | 62 | 52 | 42 | 60 | 50 |
| Conc. μg/mL at 42 hr | 60 | 73 | 95 | 84 | 79 | 81 |

Patches containing 20% Transcutol® demonstrate improved transdermal delivery of opipramol (FIG. 6). Furthermore, patches comprising the combination of Eudragit® and Transcutol® have reduced transdermal delivery of opipramol in comparison to patches without Eudragit®.

### Example 4

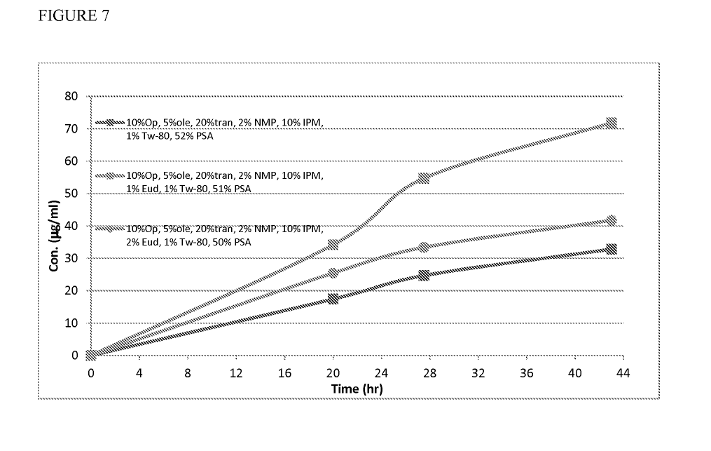

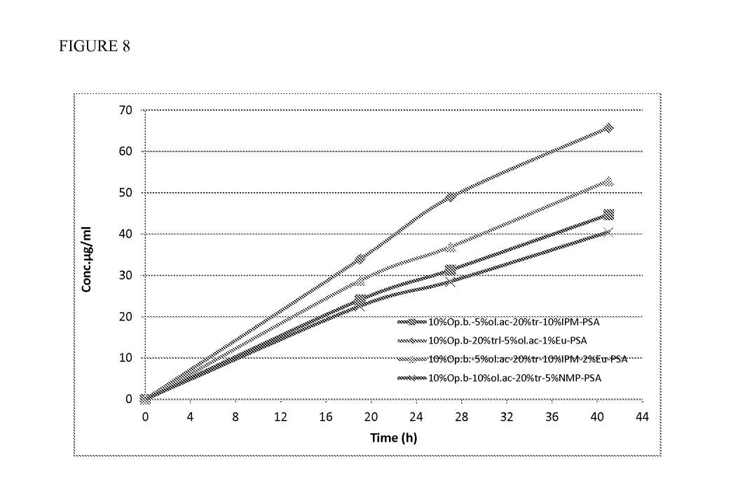

FIGS. 7 and 8 depict the effect of Eudragit® compounds on the transdermal delivery of opipramol. The effects of different concentrations of Eudragit® RL100 on the transdermal delivery of opipramol through full thickness pig skin were evaluated using the Franz Cell delivery system. Dry patches containing opipramol, oleic acid, Transcutol®, Eudragit® RL100, NMP, IPM, Tween® 80 and PSA were prepared (formulations 25-31). Samples were collected from the receiver cell at 19, 21, 27, 29 and 41 hours after application of the formulation to the skin. The amount of opipramol compounds in the receiver cell fluid was determined using a spectrophotometer at 280 nm.

FIGS. 7 and 8, and Table 7 (corresponding to FIG. 7) and Table 8 (corresponding to FIG. 8) indicate that formulations containing 1% Eudragit® RL100 showed superior transdermal delivery of opipramol through pig skin, ex vivo.

TABLE 7

| | 25 | 26 | 27 |
|---|---|---|---|
| Opipramol base % | 10 | 10 | 10 |
| Oleic acid % | 5 | 5 | 5 |
| Transcutol ® % | 20 | 20 | 20 |
| NMP % | 2 | 2 | 2 |
| IPM % | 10 | 10 | 10 |
| Eudragit ® RL100% | | 1 | 2 |
| Tween ® 80% | 1 | 1 | 1 |
| PSA (dry) % | 52 | 51 | 50 |
| Conc. μg/mL at 42 hr | 30 | 70 | 40 |

TABLE 8

| | 28 | 29 | 30 | 31 |
|---|---|---|---|---|
| Opipramol base % | 10 | 10 | 10 | 10 |
| Transcutol ® % | 20 | 20 | 20 | 20 |
| Oleic acid % | 5 | 5 | 5 | 5 |
| NMP | | | | 5 |
| IPM % | 10 | 10 | 10 | 10 |
| Tween ® 80% | 0.2 | 0.2 | 0.2 | 0.2 |
| Eudragit ® RL100% | | 1 | 2 | |
| PSA (dry) % | 54.8 | 53 | 53 | 53 |
| Conc. μg/mL at 42 hr | 45 | 67 | 52 | 40 |

### Example 5

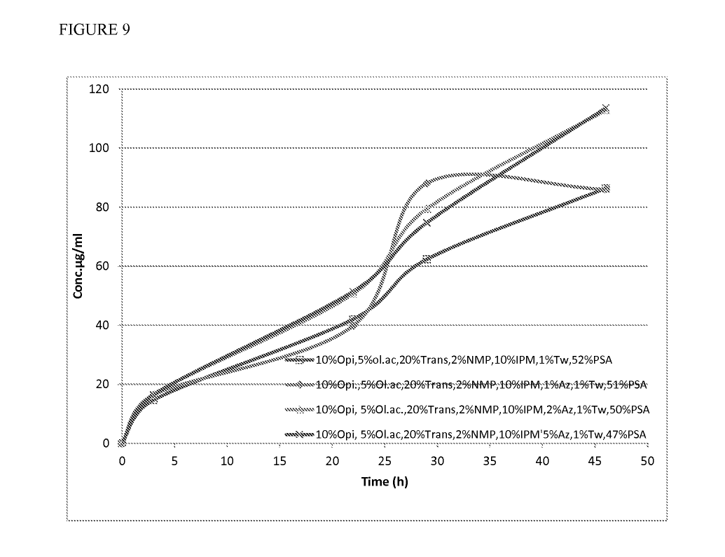

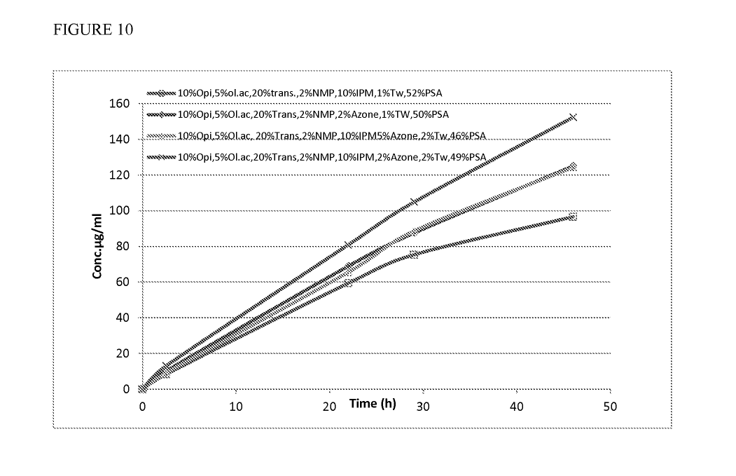

FIGS. 9 and 10 depict the effect of Azone® on the transdermal delivery of opipramol. The effects of different concentrations of Azone® on the transdermal delivery of opipramol through full thickness pig skin were evaluated using the Franz Cell delivery system. Dry patches containing opipramol, oleic acid, Transcutol®, Azone®, NMP, IPM, Tween® 80 and PSA were prepared (formulations 32-39). Samples were collected from the receiver cell at 22, 24, 27, 29, and 46 hours after application of the formulation to the skin. The amount of opipramol compounds in the receiver cell fluid was determined using a spectrophotometer at 280 nm.

FIGS. 9 and 10, and Table 9 (corresponding to FIG. 9) and Table 10 (corresponding to FIG. 10) indicate that patches containing 2% Azone® have higher transdermal delivery capabilities through pig skin ex vivo in comparison to patches containing 1% or 5% Azone®.

TABLE 9

| | 32 | 33 | 34 | 35 |
|---|---|---|---|---|
| Opipramol base % | 10 | 10 | 10 | 10 |
| Oleic acid % | 5 | 5 | 5 | 5 |
| Transcutol ® % | 20 | 20 | 20 | 20 |
| NMP % | 2 | 2 | 2 | 2 |
| IPM % | 10 | 10 | 10 | 10 |
| Azone ® % | | 1 | 2 | 5 |
| Tween ® 80% | 1 | 1 | 1 | 1 |
| PSA (dry) % | 52 | 51 | 50 | 47 |
| Conc. μg/mL at 42 hr | 85 | 85 | 115 | 115 |

TABLE 10

| | 36 | 37 | 38 | 39 |
|---|---|---|---|---|
| Opipramol base % | 10 | 10 | 10 | 10 |
| Oleic acid % | 5 | 5 | 5 | 5 |
| Transcutol ® % | 20 | 20 | 20 | 20 |
| NMP % | 2 | 2 | 2 | 2 |
| IPM % | 10 | 10 | 10 | 10 |
| Azone ® % | | 2 | 5 | 2 |
| Tween ® 80% | 1 | 1 | 2 | 2 |
| PSA (dry) % | 52 | 50 | 46 | 49 |
| Conc. μg/mL at 42 hr | 95 | 125 | 125 | 150 |

FIGS. 9 and 10 unexpectedly demonstrate that the combination of 2% Azone® with 2% Tween® 80 significantly improves the transdermal delivery of opipramol in comparison to other patches described in Tables 9 and 10.

### Example 6

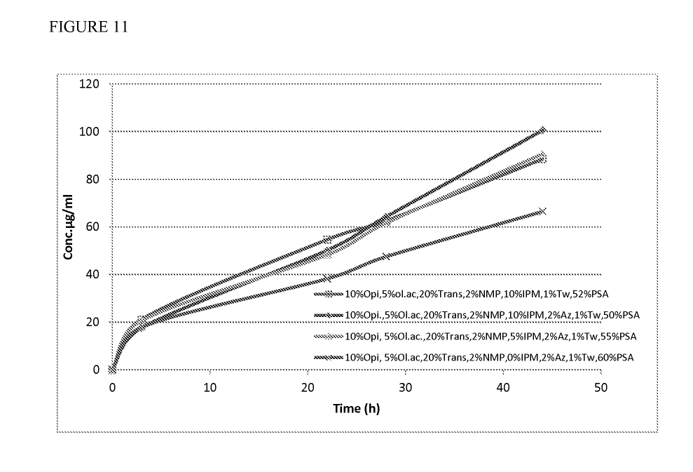

FIG. 11 depicts the effect of IPM on the transdermal delivery of opipramol. The effects of different concentrations of IPM on the transdermal delivery of opipramol through full thickness pig skin were evaluated using the Franz Cell delivery system. Dry patches containing opipramol, oleic acid, Transcutol®, NMP, IPM, Tween® 80, and PSA were prepared (formulations 40-43 in Table 11). Samples were collected from the receiver cell at 22, 28, and 44 hours after application of the formulation to the skin. The amount of opipramol compounds in the receiver cell fluid was determined using a spectrophotometer at 280 nm.

TABLE 11

| | 40 | 41 | 42 | 43 |
|---|---|---|---|---|
| Opipramol base % | 10 | 10 | 10 | 10 |
| Oleic acid % | 5 | 5 | 5 | 5 |
| Transcutol ® % | 20 | 20 | 20 | 20 |
| NMP % | 2 | 2 | 2 | 2 |
| IPM % | 10 | 10 | 5 | 0 |
| Azone ® % | | 2 | 2 | 2 |
| Tween ® 80% | 1 | 1 | 1 | 1 |
| PSA (dry) % | 52 | 50 | 55 | 60 |
| Conc. μg/mL at 42 hr | 90 | 100 | 90 | 65 |

FIG. 11 shows that patches containing 10% IPM have an improved transdermal delivery of opipramol. Furthermore, patches including 10% IPM and 2% Azone® have significantly improved transdermal delivery of opipramol in comparison to patches without IPM or different concentration of Azone®.

### Example 7

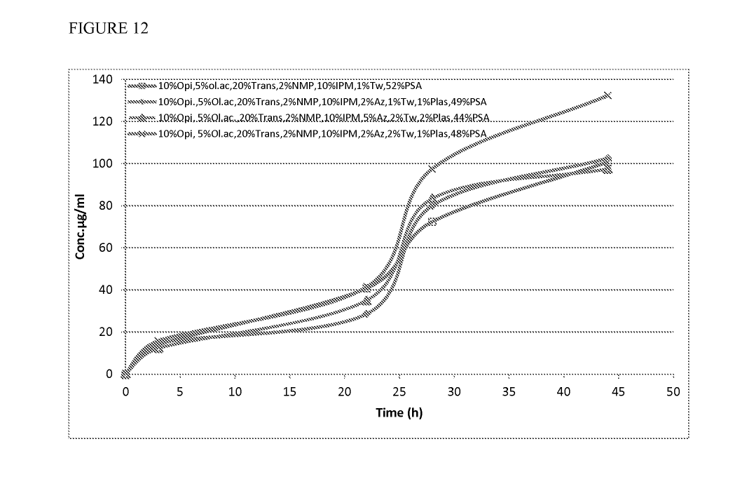

FIG. 12 depicts the effect of Plasdone™ S-630 on the transdermal delivery of opipramol. The effects of different doses of Plasdone™ S-630 on the transdermal delivery of opipramol through full thickness pig skin were evaluated using the Franz Cell delivery system. Dry patches containing opipramol, oleic acid, Transcutol®, NMP, IPM, Plasdone™ S-630, Tween® 80 and PSA were prepared (formulations 44-47). Samples were collected from the receiver cell at 22, 28, and 44 hours after application of the formulation to the skin. The amount of opipramol compounds in the receiver cell fluid was determined using a spectrophotometer at 280 nm. From FIG. 12 it can be seen that patches containing 1% Plasdone™ S-630 demonstrate an improved transdermal delivery of opipramol.

TABLE 12

| | 44 | 45 | 46 | 47 |
|---|---|---|---|---|
| Opipramol base % | 10 | 10 | 10 | 10 |
| Oleic acid % | 5 | 5 | 5 | 5 |
| Transcutol ® % | 20 | 20 | 20 | 20 |
| NMP % | 2 | 2 | 2 | 2 |
| IPM % | 10 | 10 | 10 | 10 |
| Azone ® % | | 2 | 5 | 2 |
| Tween ® 80% | 1 | 1 | 2 | 2 |
| Plasdone ™ S-630% | | 1 | 2 | 1 |
| PSA (dry) % | 52 | 49 | 44 | 48 |
| Conc. μg/mL at 42 hr | 100 | 100 | 100 | 135 |

Compositions containing 1% Plasdone™ S-630 with 2% Azone® and 2% Tween® 80 significantly improve opipramol transdermal delivery ex vivo (FIG. 12).

### Example 8

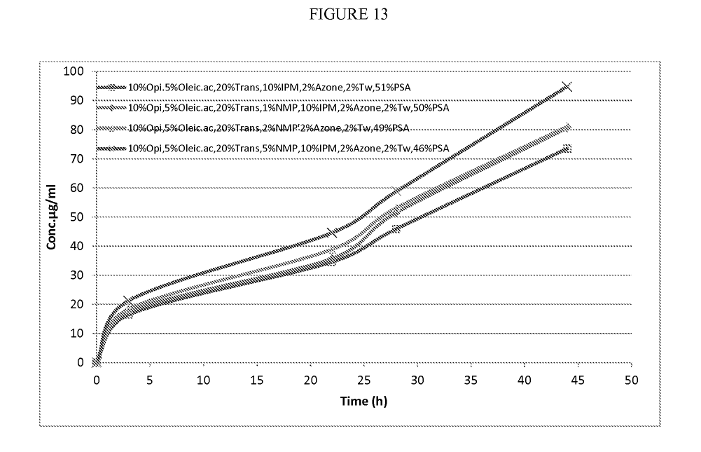

FIG. 13 depicts the effect of NMP on the transdermal delivery of opipramol. The effects of different concentrations of NMP on the transdermal delivery of opipramol through full thickness pig skin were evaluated using the Franz Cell delivery system. Dry patches containing opipramol, oleic acid, Transcutol®, NMP, IPM, Tween® 80 and PSA were prepared (formulations 48-51). Samples were collected from the receiver cell at 22, 28, and 44 hours after application of the formulation to the skin. The amount of opipramol compounds in the receiver cell fluid was determined using a spectrophotometer at 280 nm.

TABLE 13

| | 48 | 49 | 50 | 51 |
|---|---|---|---|---|
| Opipramol base % | 10 | 10 | 10 | 10 |
| Oleic acid % | 5 | 5 | 5 | 5 |
| Transcutol ® % | 20 | 20 | 20 | 20 |
| NMP % | | 1 | 2 | 5 |
| IPM % | 10 | 10 | 10 | 10 |
| Azone ® % | 2 | 2 | 2 | 2 |
| Tween ® 80% | 2 | 2 | 2 | 2 |
| PSA (dry) % | 51 | 50 | 49 | 46 |
| Conc. μg/mL at 42 hr | 63 | 80 | 80 | 95 |

From FIG. 13 one can see that patches containing 5% NMP improve transdermal delivery of opipramol.

### Example 9

In this experiment, the purpose was to determine the transdermal delivery of opipramol in pigs. Test patches containing 10% opipramol base, 5% oleic acid, 20% Transcutol®, 2% NMP, 10% IPM, 2% Azone®, 2% Tween® 80, and 49% PSA were applied for 48-hour period to the ears of female pigs (85-90 kg). Blood samples were collected at pre-determined time points and plasma levels of opipramol were analyzed by HPLC-UV.

### Patch Application

Prior to application, each pig (Swine/Landrace× large White) was anesthetized by intravenous injection of Midazolam/Ketamin. Opipramol patch(es) was applied on to area of 6 cm×6.5 cm directly to the ear skin of the animal. The patch was secured with skin clips at the edges of the patch and adhesive tape.

The Test Formulation was applied for a period of about 48 hours. At the end of the exposure period, residual formulation was removed using tap water without altering the existing response or the integrity of the epidermis. The margins of the test site were marked with non-irritating and non-erasable ink in order to facilitate the subsequent observation sessions.

### Blood Collection

Blood samples, each of approximately 9-10 mL, were collected from the external jugular vein via the intravenous cannula into vials containing EDTA. Samples were collected at time points: Day 0: Prior to application of patch(es) and 7-8 & hours post application. Day 1: 23-24, 27-28 and 31-32 hours post application of patch(es). Day 2: 47-48 hours post application of patch(es), prior to removal of patch, 2-3 and 7-8 hours post removal of patch(es). Day 3: 24 hours post removal of patch(es).

Immediately after collection, whole blood samples were placed on ice and centrifuged at 4° C., 3,500 rpm for 7 min. Plasma, total of about 5 mL, was transferred into three pre-labeled tubes (1.6 mL/tube). All samples were stored at −20° C. until further analysis.

### Plasma Concentration

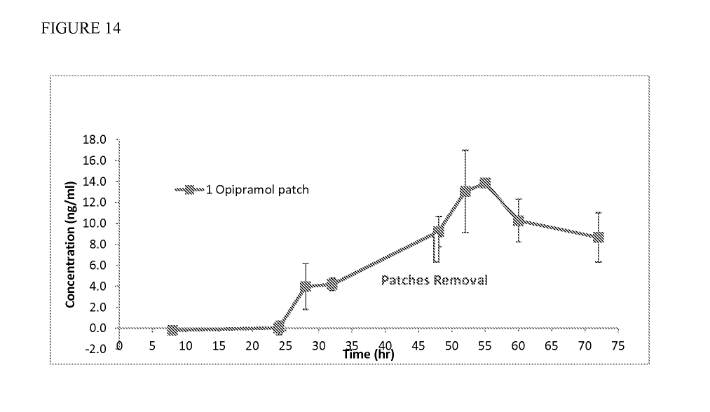

The concentration of opipramol in the plasma was measured. The plasma levels of the newly developed test formulations of opipramol could be detected following transdermal administration (patch) for 48-hour period in female pigs. The level of opipramol was elevated in a time-related manner. Several hours after the patches were removed, a reduction in the plasma levels occurred (FIG. 14).

### Example 10

This experiment was performed to determine the plasma levels of opipramol following continuous transdermal administration of opipramol patch for 72 hours. Test patches containing 10% opipramol base, 5% oleic acid, 20% Transcutol®, 2% NMP, 10% IPM, 2% Azone®, 2% Tween® 80, and 49% PSA were applied for 48-hour period to the ears of female pigs. Blood samples were collected at pre-determined time points and plasma levels of opipramol were analyzed by HPLC-UV.

### Jugular Catheterization

Prior to the first dosing session animals were subjected to intravenous jugular cannulation under general anesthesia, with an indwelling cannula subsequently exteriorized and affixed to the skin.

### Pre-Test Preparation

Prior to study initiation, the ears of the animals were shaved with care to avoid abrading the skin, and the skin was thoroughly cleansed with Septiscrub and 70% ethanol.

### Patch Application

Prior to patch adhesion, animals were anesthetized. Small animals (pigs around 32 kg) were anesthetized with 0.5 mL Ketamine and 1.5 mL Midazolam and large animals (pigs around 80 kg) were anesthetized with 1 mL Ketamine and 3 mL Midazolam. Opipramol patch was applied on to area of about 25 cm²-35 cm², directly to the ear skin. The patch was secured with adhesive tape. The margins of the test site were marked with non-irritating and non-erasable ink in order to facilitate the subsequent observation sessions.

The Test Formulation was applied for a period of 72 hours (three days) or 168 hours (one week). At the end of the exposure period, residual formulation was removed using tap water without altering the existing response or the integrity of the epidermis. The margins of the test site were marked with non-irritating and non-erasable ink in order to facilitate the subsequent observation sessions.

### Blood Collection

Blood samples, each of approximately 6-7 mL, were collected from the external jugular vein via the intravenous cannula into vials containing EDTA.

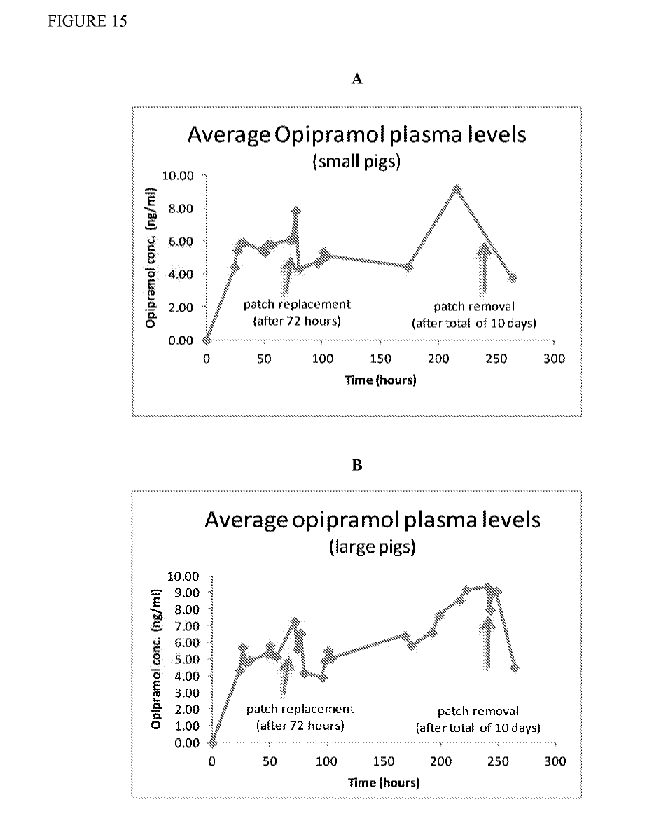

Immediately after collection, whole blood samples were centrifuged at 4° C., 3,500 rpm for 7 min. Plasma, total of about 3.5 mL, was transferred into two pre-labeled tubes (1.7 mL/tube). All samples were stored at −20° C. until further analysis. The concentration of opipramol plasma levels elevated with time and reached peak (5-6 ng/mL) about 26-29 hours following patch application (FIGS. 15 A and B). No major change in concentration was noted following patch replacement (after 3 days) and the levels remained between 3.85-10.54 ng/mL with minor fluctuations until patch removal (10 days from application). After removal of the 2^{nd} patch (applied for 1 week) the concentration of plasma opipramol decreased.

Opipramol was detected in the plasma for at least 7 days following patch application. No significant difference was noted between the opipramol plasma levels of the small vs. the larger pigs.

### Example 11

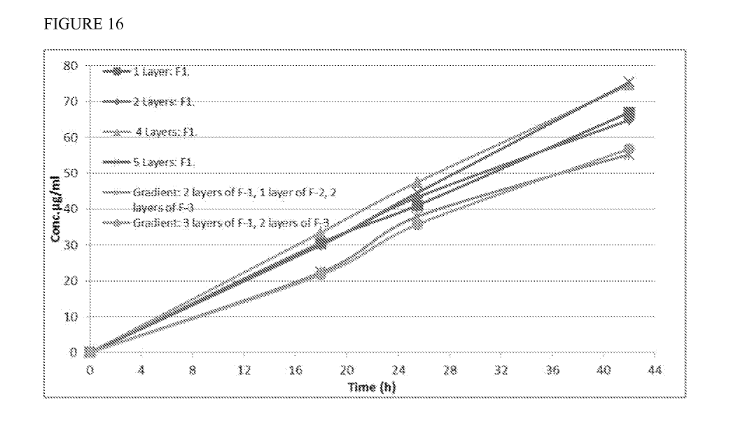

The transdermal delivery of patches containing multiple layers was also examined. FIG. 16 shows the delivery rates of opipramol by patches containing differing types of layers (F1-F3 in Table 14 below) each varying slightly in ingredient concentrations as well as differing numbers of layers.

The most rapid delivery of opipramol was achieved by a patch containing 4 layers of F1. Patches with opipramol gradient perform similarly in the transdermal delivery of opipramol compared with patches comprising 10% opipramol.

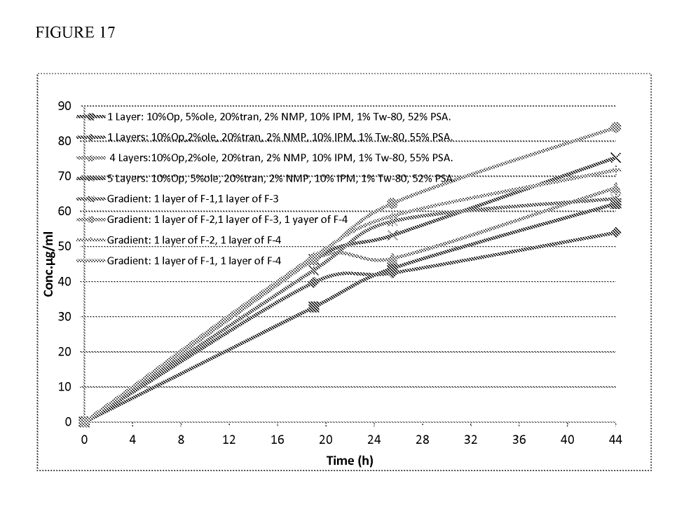

FIG. 17 shows the delivery rates of opipramol by patches containing differing types of layers (F1-F4 in Table 15 below) each varying slightly in ingredient concentrations as well as differing numbers of layers. The most rapid delivery was achieved with a patch containing three layers (F2, F3, and F4). Patches comprising a gradient of oleic acid show improved transdermal delivery of opipramol over patches comprising a uniform concentration of oleic acid.

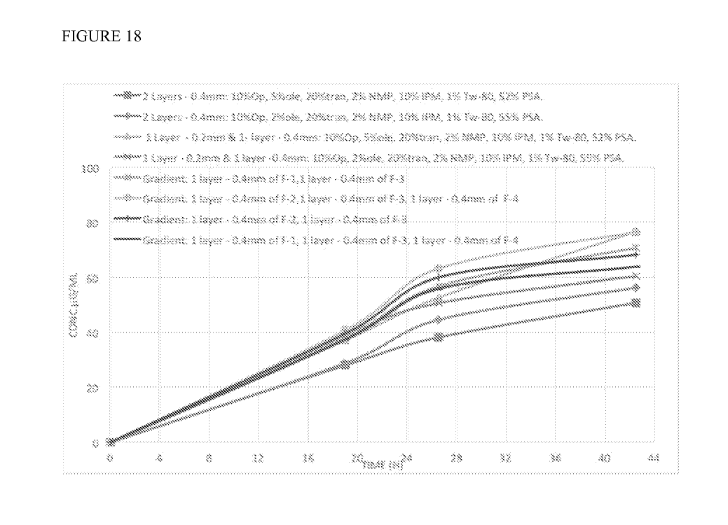

FIG. 18 shows the delivery rates of opipramol by patches containing differing types of layers (F1-F4 in Table 16 below) each varying slightly in ingredient concentrations as well as differing numbers of layers. The most rapid delivery was achieved with a patch containing three layers of 0.4 mm thickness (F2, F3, and F4). Patches comprising 3 layers with a gradient of opipramol and a gradient of oleic acid show improved transdermal delivery in comparison with patches comprising a single concentration of oleic acid (Patch 6 vs. Patch 8). Furthermore, patches comprising a gradient in the thickness of the membranes show higher transdermal delivery of opipramol in comparison with patches without the gradient.

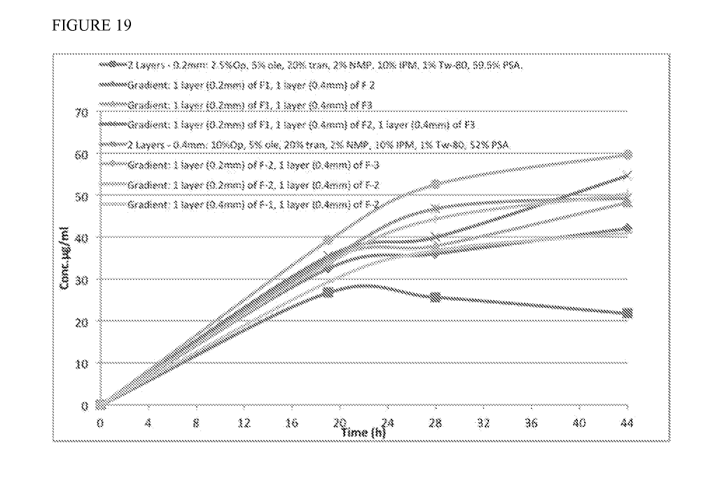

FIG. 19 shows the delivery rates of opipramol by patches containing differing types of layers (F1-F3 in Table 17 below) each varying slightly in ingredient concentrations as well as differing numbers of layers. The most rapid delivery was achieved with a patch containing two layers (F2 and F3). The concentration of opipramol increased linearly over the first few hours for all entries followed by gradual slowing of the rate of increase. Patches including a gradient of IPM show an improved transdermal delivery of opipramol in comparison to patches without the IPM gradient (Patch 6 vs. Patch 7). Furthermore, patches including a thickness membrane gradient show an improved transdermal delivery of opipramol (Patch 6 vs. Patch 5).

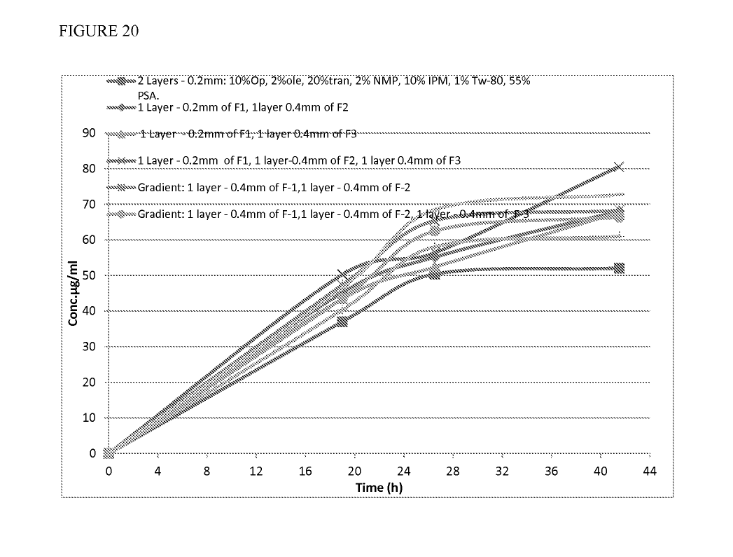

FIG. 20 shows the delivery rates of opipramol by patches containing differing types of layers (F1-F3 in Table 18 below) each varying slightly in ingredient concentrations as well as differing numbers of layers. The patches performed similarly, giving a rapid, linear increase in opipramol concentration followed by very gradual increase in concentration after 20 hours from the beginning of the experiment.

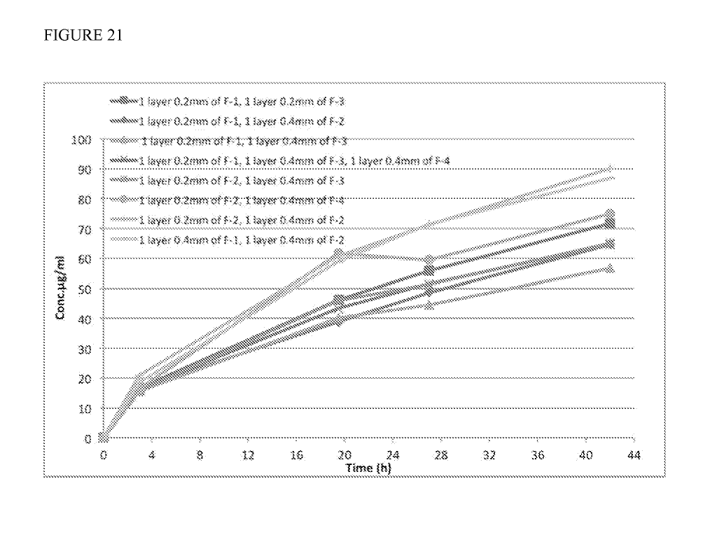

FIG. 21 shows the delivery rates of opipramol by patches containing differing types of layers (F1-F4 in Table 19 below) each varying slightly in ingredient concentrations as well as differing numbers of layers. The most rapid increase in opipramol concentration was afforded by a patch containing a 0.2 mm layer of F2 and a 0.4 layer of F2. Patches comprising Eudragit® have an improved transdermal delivery of opipramol over the other patches.

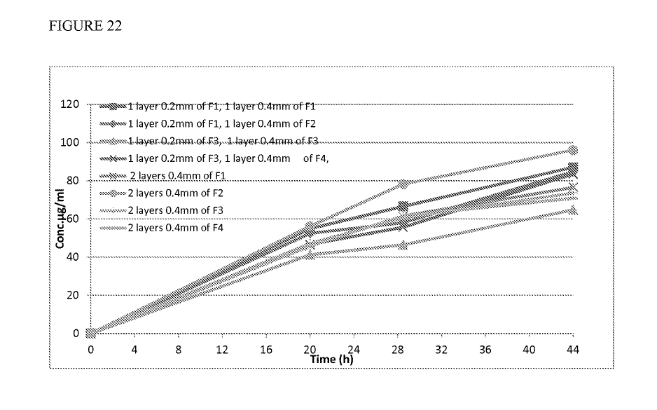

FIG. 22 shows the delivery rates of opipramol by patches containing differing types of layers (F1-F4 in Table 20 below) each varying slightly in ingredient concentrations as well as differing numbers of layers. A patch containing two 0.4 mm layers of comprising 5% oleic acid afforded the most rapid increase in opipramol concentration. Patches dried for 40 min at 70° C. show an improved transdermal delivery of opipramol in comparison to patches dried for 15 min at 80° C. (Patch 6 vs. Patch 8 and Patch 1 vs. Patch 3). Furthermore, patches comprising a thickness gradient show improved transdermal delivery of opipramol versus patches without thickness gradient (Patch 1 vs. Patch 5).

### EQUIVALENTS

Those skilled in the art will recognize, or be able to ascertain using no more than routine experimentation, many equivalents to the specific embodiments of the present disclosure described herein. Such equivalents are intended to be encompassed by the following claims.

### INCORPORATION BY REFERENCE

The entire contents of all patents, published patent applications, websites, and other references cited herein are hereby expressly incorporated herein in their entireties by reference.

1. A transdermal drug composition for the transdermal delivery of opipramol to a patient, the drug composition comprising:
a. a plasticizer;
b. a penetration enhancer;
c. a pressure-sensitive adhesive (PSA); and
d. opipramol or a pharmaceutically acceptable salt thereof,
wherein said drug composition can form an adhesive layer.

2. The transdermal drug composition of claim 1, further comprising a hydrophilic polymer selected from the group consisting of a polymethacrylate polymer and a polyvinylpyrrolidone polymer, or a combination thereof.

3-4. (canceled)

5. The transdermal drug composition of claim 1, wherein the plasticizer is selected from the group consisting of a fatty alcohol, a citric acid alkyl ester, a glycerol ester, phthalic acid alkyl ester, a sebacic acid alkyl ester, a sucrose ester, a sorbitan ester, an acetylated monoglyceride, a polyol, a fatty acid of 4-15 carbons, a fatty acid ester, a poloxamer, a mono- or di-glyceride of edible fats or oils, a glyceride, a polyethylene glycol (PEG), a sorbitan ester, a polysorbate, a disaccharide, and 2-(2-ethoxyethoxy)ethanol, or a combination thereof.

6. (canceled)

7. The transdermal drug composition of claim 1, wherein said penetration enhancer is selected from the group consisting of a C₁-C₁₂ alcohol or ester, a C₂-C₃₀ diol, a C₃-C₃₀ polyol, a fatty alcohol, a fatty acid, a fatty acid ester, a polyoxyethylene fatty acid ester, a cyclic or N,N-dimethyl amide, a sorbitan monoester, a polyethylene glycol ether, a biodegradable cyclic urea, a polysaccharide, a terpene or essential oil, a surfactant, a sulfoxide, and a fatty acid or polyoxyethylene triglyceride, or a combination thereof.

8. (canceled)

9. The transdermal drug composition of claim 1, wherein the penetration enhancer is selected from the group consisting of propylene glycol, N-methylpyrrolidone (NMP), polyoxypropylene (15) stearyl ether, dimethylisosorbate (DMI), 1-dodecylazacycloheptane-2-one, sorbitan laurate, polysorbate 20, polysorbate 40, polysorbate 60, polysorbate 80, oleic acid, 2-(2-ethoxyethoxy)ethanol, and isopropyl myristate (IPM), or a combination thereof.

10. The transdermal drug composition of claim 1, wherein the PSA is selected from the group consisting of an acrylic-based, a rubber-based, a silicone-based, a polyurethane-based, a polyester-based, and a polyether-based adhesive, or a combination thereof.

11. The transdermal drug composition of claim 1, wherein the PSA is an acrylate copolymer or a polyhydrocarbon copolymer.

12-13. (canceled)

14. The transdermal drug composition of claim 1, wherein the opipramol is selected from the group consisting of opipramol tartrate, opipramol succinate, opipramol fumarate, opipramol mesylate, opipramol lactate, opipramol oleate, and opipramol azylate.

15. The transdermal drug composition of claim 1, wherein the opipramol is opipramol free base.

16. The transdermal drug composition of claim 1, wherein the opipramol is from about 1 to about 25%, or about 5% to about 20%, or about 7.5% to about 12.5% w/w based on the total weight of the composition.

17-20. (canceled)

21. The transdermal drug composition of claim 16, wherein the opipramol is dissolved in the composition.

22. The transdermal drug composition of claim 1, comprising:
0% to about 3% w/w of a hydrophilic polymer;
about 0.001% to about 30% w/w of the plasticizer;
about 5% to about 25% w/w of one or more penetration enhancers;
about 40% to about 80% w/w of the PSA; and
about 1% to about 25% w/w of the opipramol or a pharmaceutically acceptable salt thereof.

23-26. (canceled)

27. The transdermal drug composition of claim 1, wherein:
the plasticizer is 2-(2-ethoxyethoxy)ethanol and comprises about 20% w/w of the composition; and/or the composition comprises at least one of the following penetration enhancers: up to about 5% w/w oleic acid, up to about 10% w/w polyoxypropylene (15) stearyl ether, up to about 10% w/w DMI, up to about 10% w/w IPM, and up to about 2% w/w polysorbate 80; and
the PSA comprises about 53 to about 60% w/w of the composition.

28. The transdermal drug composition of claim 1, wherein:
the plasticizer is 2-(2-ethoxyethoxy)ethanol and comprises about 20% w/w of the composition;
the penetration enhancer comprises about 1% to about 2% w/w polysorbate 80 and optionally comprises up to about 5% w/w NMP, up to about 5% w/w oleic acid, and up to about 5% w/w 1-dodecylazacycloheptane-2-one;
and the PSA comprises about 41% to about 58% w/w of the composition; and
optionally further comprising up to about 2% w/w of a hydrophilic polymer, wherein the hydrophilic polymer is a 60:40 random copolymer of vinyl pyrrolidone and vinyl acetate.

29. (canceled)

30. The transdermal drug composition of claim 1, wherein:
the plasticizer is 2-(2-ethoxyethoxy)ethanol and comprises about 20% w/w of the composition;
the penetration enhancer is a mixture of about 1% to about 5% w/w azelaic acid, about 10% w/w IPM, and about 0.2% to about 5% w/w polysorbate 80; and
the PSA comprises about 49% to about 54.8% w/w of the composition.

31. The transdermal drug composition of claim 1, wherein:
the plasticizer is 2-(2-ethoxyethoxy)ethanol and comprises about 20% w/w of the composition;
the penetration enhancer is a mixture of about 5% w/w oleic acid, about 10% w/w IPM, and about 0.2% to about 5% w/w polysorbate 80; and
the PSA comprises about 49% to about 54.8% w/w of the composition.

32-33. (canceled)

34. The transdermal drug composition of claim 1, wherein:
the plasticizer is 2-(2-ethoxyethoxy)ethanol and comprises about 20% w/w of the composition;
the penetration enhancer comprises about 5% w/w oleic acid, up to about 5% w/w NMP, about 10% w/w IPM, and about 0.2% w/w polysorbate 80; and
the PSA comprises about 53% to about 54.8% w/w of the composition.

35-36. (canceled)

37. The transdermal drug composition of claim 1, wherein:
the plasticizer is 2-(2-ethoxyethoxy)ethanol and comprises about 20% w/w of the composition, or is oleyl alcohol and comprises about 10% w/w of the composition; the penetration enhancer optionally comprises up to about 5% w/w oleic acid and up to about 10% w/w IPM; and
the PSA comprises about 59% to about 85% w/w of the composition.

38-41. (canceled)

42. The transdermal drug composition of claim 1, wherein
the plasticizer is 2-(2-ethoxyethoxy)ethanol and comprises about 5% to about 30% w/w of the composition;
the penetration enhancer is a mixture comprising about 5% to about 10% w/w oleic acid; about 2% w/w NMP; about 5% to about 10% w/w IPM; and about 1% w/w surfactant; and the PSA comprises about 52% to about 77% w/w of the composition.

43-44. (canceled)

45. A transdermal delivery device for the transdermal delivery of opipramol comprising:
a. an inert layer detachable when used;
b. at least one adhesive layer comprising a transdermal drug composition of claim 1, wherein the adhesive layer is directly affixed to a surface of the inert layer; and
c. a backing layer, coated over the adhesive layer.

46-52. (canceled)

53. A method of treating a patient having a disorder selected from the group consisting of central nervous system (CNS) disorders, peripheral nervous system disorders, factitious disorders, somatoform disorders, inflammatory disorders, and pain-related disorders, said method comprising the steps of:
a. providing a transdermal delivery device according to claim 45; and
b. placing an adhesive layer of the device against the skin of the patient, thereby providing an amount of opipramol effective to treat the disorder.

54. A method of preventing, treating, or suppressing tobacco or nicotine dependence or usage in a patient, said method comprising the steps of:
a. providing a transdermal delivery device according to claim 45; and
b. placing the adhesive layer of the device against the skin of the patient, thereby providing an amount of opipramol effective to prevent, treat, or suppress the tobacco or nicotine dependence or usage.

55-60. (canceled)
